(19) United States
(12) Patent Application Publication
(10) Pub. No.: US 2004/0092581 A1
(43) Pub. Date: May 13, 2004

(54) Ratjadone derivatives for inhibiting cell growth

(76) Inventors: Arne Burzlaff, Hannover (DE); Cornelia Kasper, Hannover (DE); Thomas Scheper, Hannover (DE); Markus Kalesse, Loetzen (DE); Ulhas Bhatt, Hannover (DE); Khandavalli Chary, Hannover (DE); Eckhard Claus, Frankfurt am Main (DE); Mathias Christmann, Peine (DE); Monika Quitschalle, Hannover (DE); Winfried Beil, Hannover (DE)

Correspondence Address: Pendorff & Cutliff, 5111 Memorial Highway, Tampa, FL, 33634-7356, US

(21) Appl. No.: 10/467,876

(22) PCT Filed: Jan. 26, 2002
(86) PCT No.: PCT/EP02/00817

(30) Foreign Application Priority Data
Feb. 12, 2001 (DE) 101066473

(51) Int. Cl. A61K 31/366; C 07D 4 5/02

(52) U.S. Cl. 514/460; 549/273

(57) ABSTRACT

The invention relates to ratjadone derivatives which can stop the cell cycle of tumour cells in the G1 phase and/or exhibit a reduced cytotoxicity as ratjadone. According to the invention, the ratjadone derivatives correspond to formula (II) wherein R₁, R₂ and R₃ are selected independently from each other from the group which is made of H, CH₃ and C₂H₅, R₄ is CH₃ or C₂H₅, R₅ is H or OH and R₆ and R₇ are selected independently from each other from the group which is made of H, CH₃, C₂H₅, n-C₃H₇, and iso C₃H₇, and wherein C₁₀ is R-configured and C₁₇ is R-configured if (a) C₁₆ is R-configured and simultaneously (b) neither R₅, nor R₆, nor R₇ are H. The invention also relates to a method for synthesizing ratjadone derivatives.

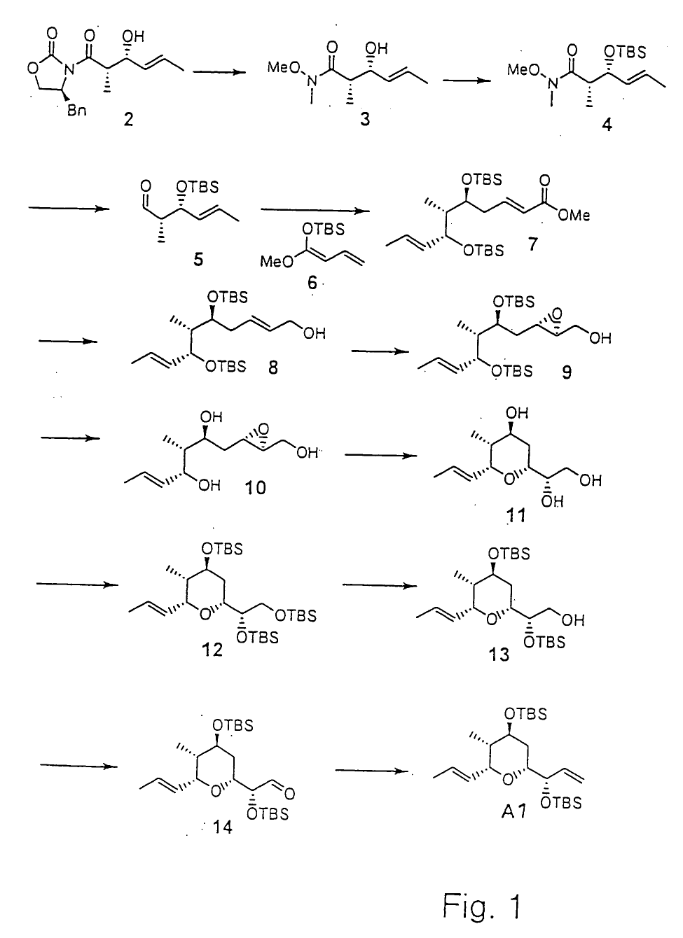

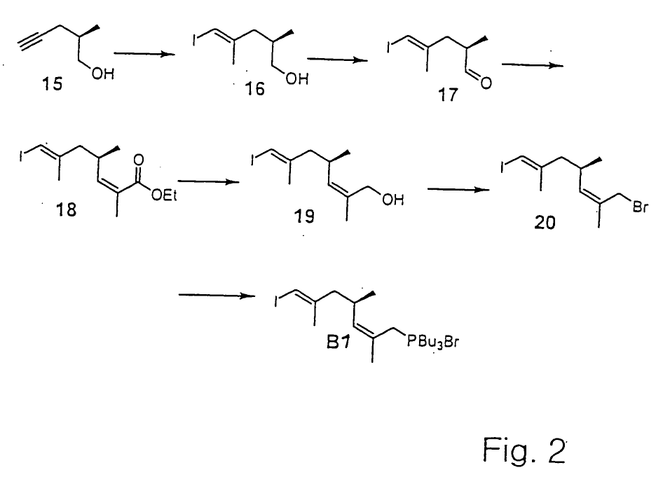

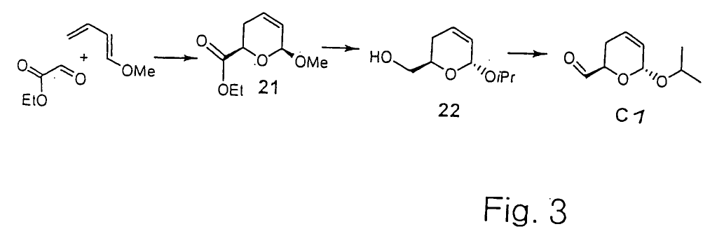

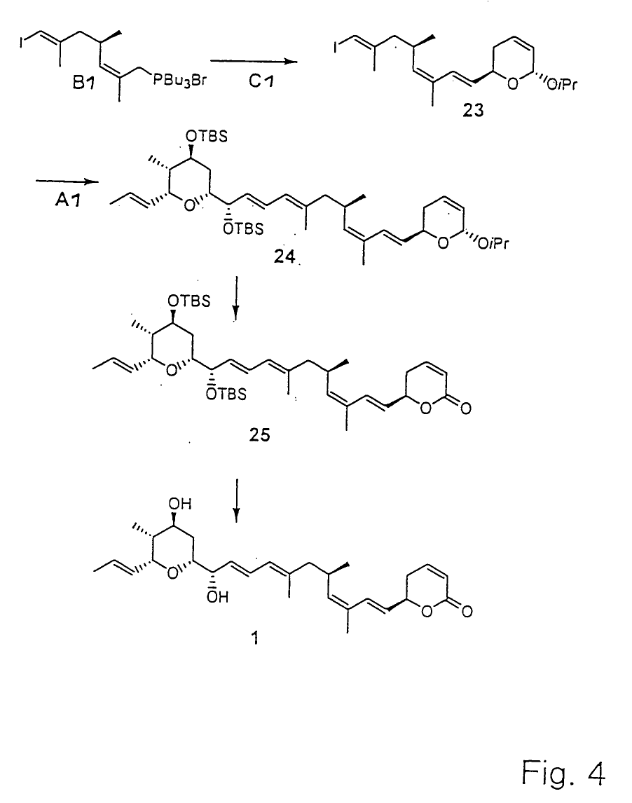

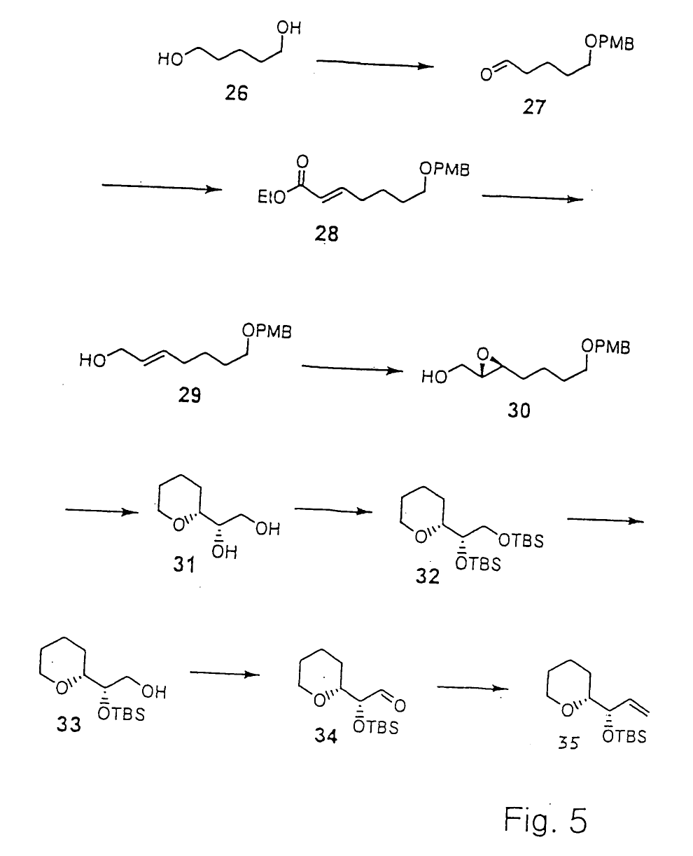

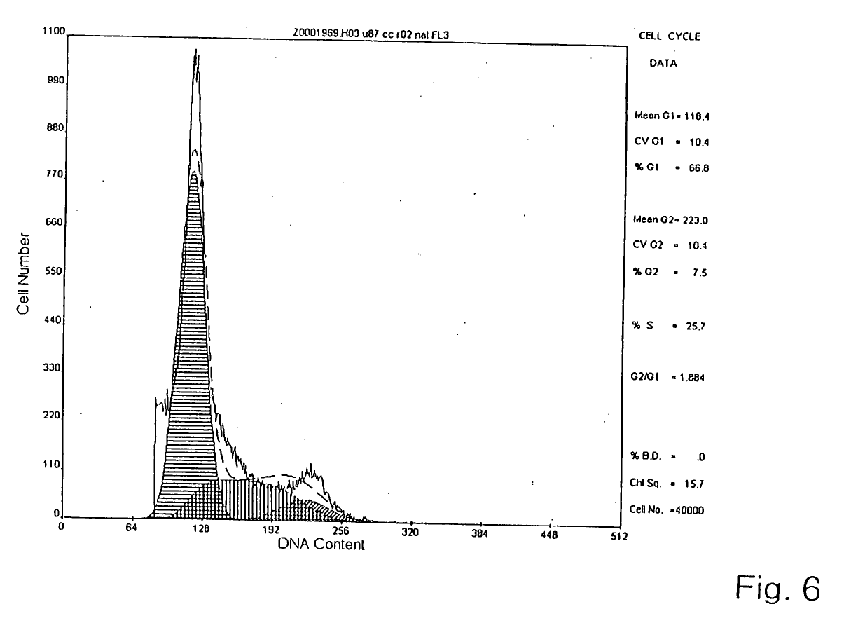

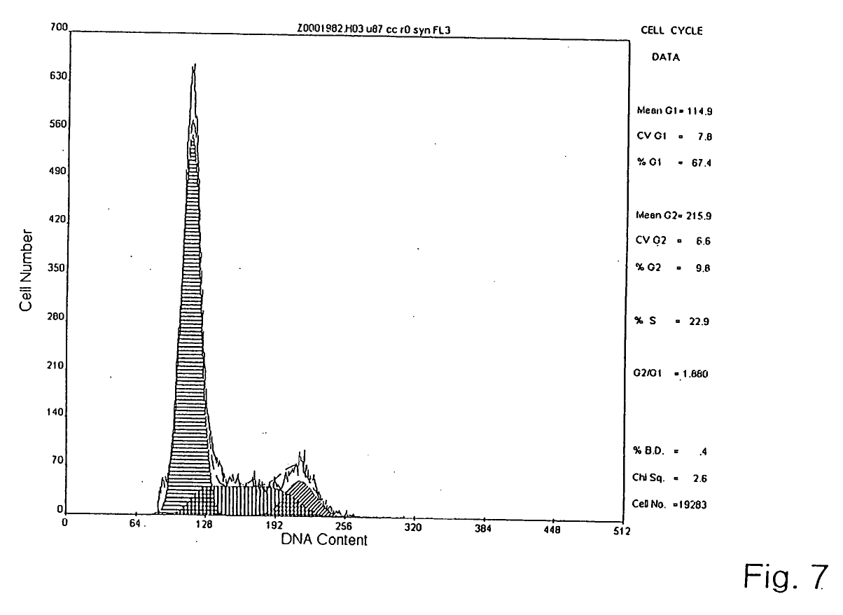

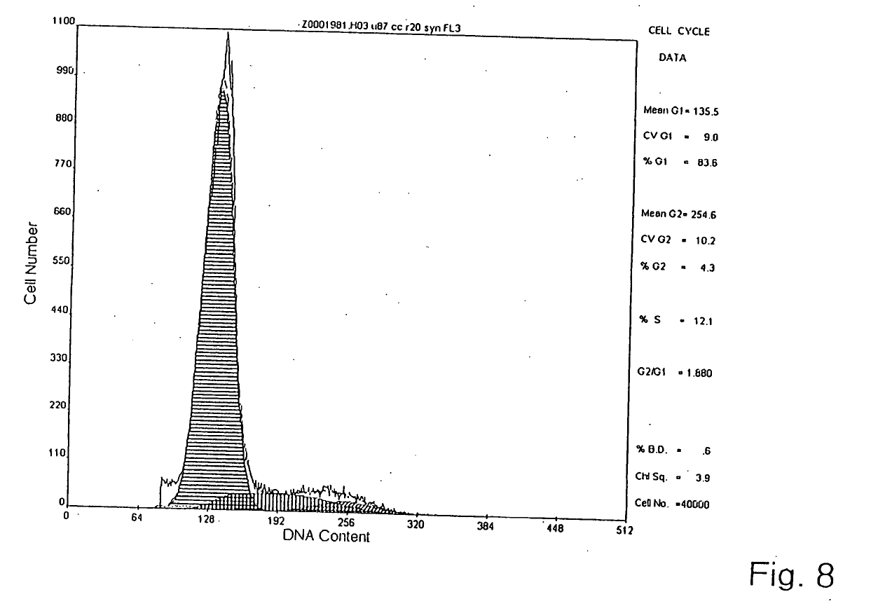

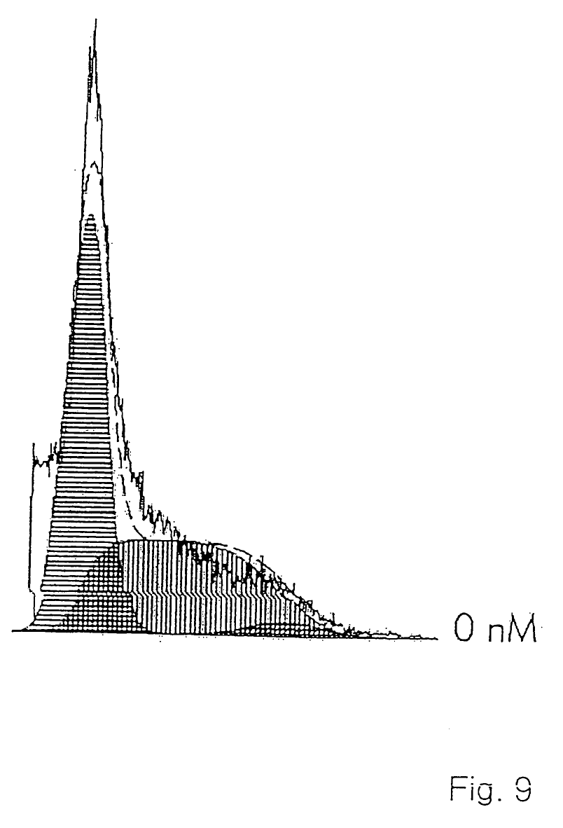

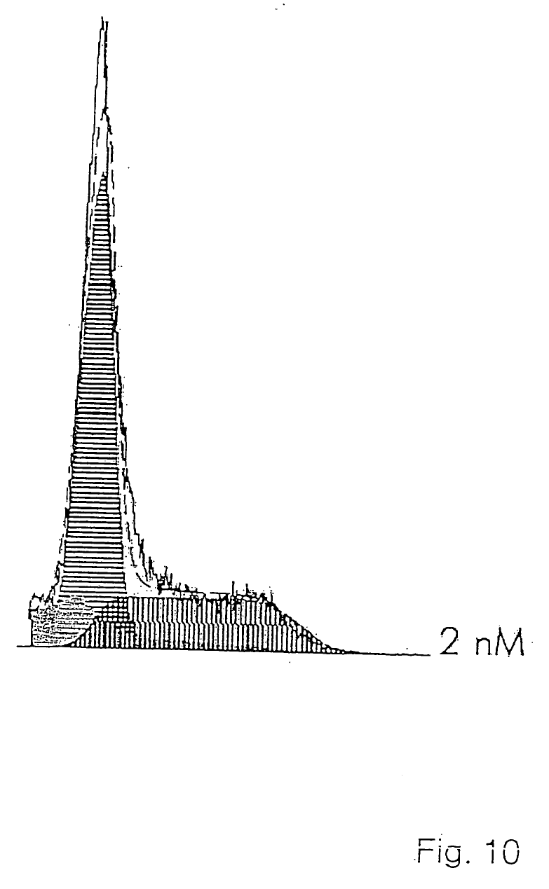

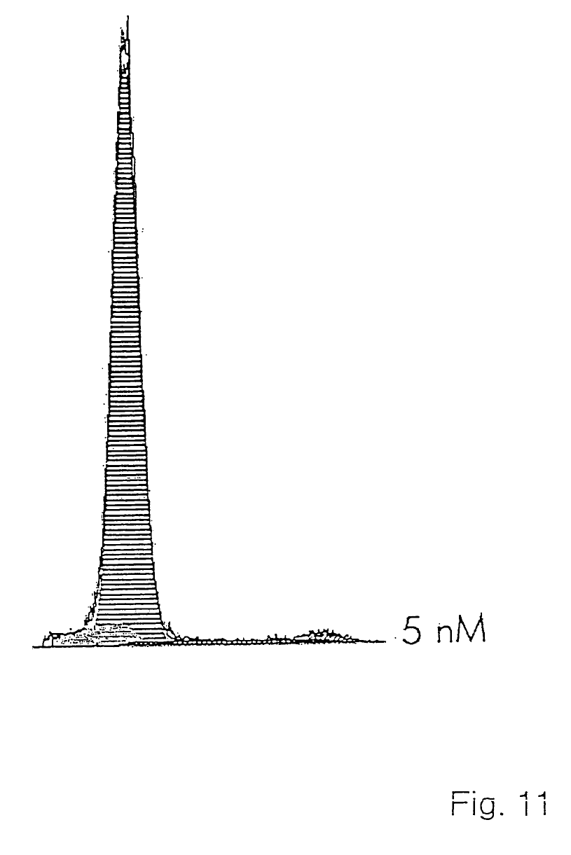

The invention relates to ratjadon derivatives and to methods for the preparation of these substances.

The naturally occurring substance ratjadon (formula I) was isolated in 1994 by Höfle et al. from the myxobacterium strain Sorangium cellulosum (So ce360) (D. Schummer, K. Gerth, H. Reichenbach, G. Höfle, Liebigs Ann., 1995, 685-688). Ratjadon inhibits the growth of HeLa cells (KB3.1) in low concentration (IC₅₀=50 pg ml⁻¹, cf. K. Gerth, D. Schummer, G. Höfle, H. Irschik, H. Reichenbach, J. Antibiot. 1995, 48, 973-976). In a publication by the Applicants (M. Christmann, U. Bhatt, M. Quitschalle, E. Claus, M. Kalesse, Angew. Chem. 2000, 112, 4535-4538) we have shown that the chiral centres C5, C10 and C16 of ratjadon are R-configured; this was not previously known.

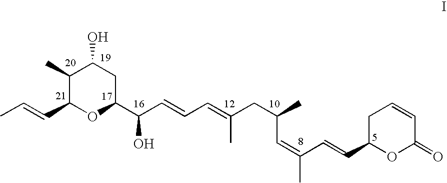

A disadvantage of ratjadon is its high cytotoxicity. In practice its applicability for the inhibition of cell growth is severely restricted since its cytotoxicity makes very accurate dosage necessary. A further disadvantageous effect, in addition to this restricted applicability, is that the synthesis of ratjadon is time-consuming and expensive, so that it appears to be uneconomic for carrying out on an industrial scale.

One aim of the present invention was, therefore, to indicate other substances that inhibit cell growth, in particular tumor cells, at similarly low concentrations as ratjadon.

A further aim was to indicate cell growth-inhibiting substances that have a lower cytotoxicity than ratjadon.

A further aim was to indicate cell growth-inhibiting substances for which the synthesis is less time-consuming than that of ratjadon.

These aims are achieved by a substance, specifically a ratjadon derivative, of the formula

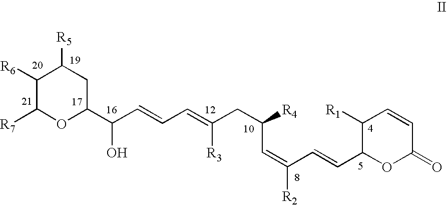

where R₁, R₂ and R₃ independently of one another are selected from the group that consists of H, CH₃ and C₂H₅, R4 is CH₃ or C₂H₅, R₅ is H or OH and R6 and R₇ independently of one another are selected from the group that consists of H, CH₃, C₂H₅, n-C₃H₇, iso-C₃H₇,

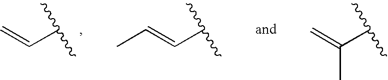

and where C10 is R-configured and C17 is R-configured if (a) C16 is R-configured and at the same time (b) neither R₅ nor R₆ nor R₇ is H.

Here the stepped line

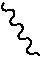

symbolizes the coupling to the relevant assigned carbon atom of the tetrahydropyran sub-unit (C19, C20 and C21 respectively).

Substances of the formula II in which the radicals R₁ to R₇ have the above meanings and with the indicated configuration restrictions are designated “substance of the formula H according to the invention” below.

The substances of the formula II according to the invention are distinguished from the naturally occurring substance ratjadon (a) at least in respect of one of the radicals R₅ to R₇ and/or (b) in respect of the configuration of the carbon atoms C16 and/or C17.

The invention is, on the one hand, based on the finding that the cytotoxicity of a substance of the formula II according to the invention is regularly lower than that of ratjadon if one or more of the radicals R₅, R₆ and R₇ is H instead of—as in the case of ratjadon—OH, methyl or propenyl. In this case the cell growth-inhibiting and cell reproduction-inhibiting effect remains essentially unchanged, so that the therapeutic range of such substances is generally greater than that of ratjadon. In the context of this text “therapeutic range” signifies the ratio between the concentration at which 50% of the cells die (cytotoxic concentration, LC₅₀), and the concentration at which the cell growth of 50% of the cells is completely inhibited, without the cells dying within 12 h (cell growth-inhibiting concentration, GI₅₀).

In the context of this text “cell growth” denotes the process of replication of the genetic material of a cell, its growth in size and its division. The term denotes in particular the transition between the G₀, G₁, S, G₂ and mitosis phases of the cell cycle or cell division cycle as well as the processes in these particular cell cycle phases. Cell growth-inhibiting substances are thus, in particular, those substances that inhibit cell reproduction and arrest the cell cycle in a phase (cell cycle arrest).

An additional advantage of substances in which one or more of the radicals R₅ to R₇ has been replaced by H lies in the fact that they can be synthesized more easily than natural ratjadon. A synthesis of these substances on an industrial scale is therefore more economic than an industrial synthesis of ratjadon.

The invention is, on the other hand, based on the finding that a substance of the formula II according to the invention has a lower cytotoxicity than ratjadon if the configuration of the carbon atoms C16 and/or C17 differs from that of ratjadon, thus if C16 is R-configured and C17 is R-configured, or C16 is S-configured and C17 is R- or S-configured. Surprisingly, it has also been found that these substances still inhibit cell growth and cell reproduction at similarly low concentrations to ratjadon.

In this context, those substances of the formula II according to the invention in which C16 is S-configured and C17 is R-configured are particularly preferred. These substances have a particularly low cytotoxicity, but are still already able to inhibit cell growth and cell reproduction at similarly low concentrations to ratjadon.

A common feature of the substances according to the invention is their ability to retard or completely to inhibit the growth of tumor cells in cell culture and/or in the living organism and to kill tumor cells within a few days when treatment is continued (in this context see Example 36 further below). In sufficient dosage, substances of the formula H according to the invention are regularly able to arrest cells in the G₁ cell cycle phase. The treatment of cells growing on a carrier surface with substances of the formula II according to the invention also regularly leads to it being possible easily to detach these cells from the carrier.

The substances of the formula II according to the invention are R-configured at C10. It has been found, surprisingly, that substances with an S-configuration at this carbon atom have a cell growth-inhibiting and cell reproduction-inhibiting action only at elevated concentrations and that the therapeutic range is reduced.

Preferably C5 is R-configured. Such substances generally have a more powerful growth-inhibiting action (i.e. they already inhibit cell growth at lower concentrations) than the corresponding epimers S-configured at C5.

The substances of the formula H according to the invention can have further chiral centres:

If R₁ is not H, the assigned carbon atom C4 is chiral.

The carbon atoms C19, C20 and C21 can likewise be chiral centres and specifically if the relevant associated radicals R₅, R₆ and R₇, respectively, are not H. In these cases it is preferable if C19 is S-configured and/or C20 is S-configured and/or C21 is R-configured. Such substances can be synthesized more easily than ratjadon and also have a powerful cell growth-inhibiting and cell reproduction-inhibiting action with a large therapeutic range.

A substance of the formula II, according to the invention in which R₅, R₆ and R₇ are each H is preferred. Because of the reduced number of chiral centres, such a substance can be prepared particularly easily and at low cost, including on an industrial scale. It also has a powerful cell growth-inhibiting and cell reproduction-inhibiting action and is less cytotoxic compared with ratjadon.

A further aim was to indicate a formulation for inhibition of the reproduction of tumor cells where one of the or the active substances of the formulation is (are) to have a cell reproduction-inhibiting action in a similarly low concentration to or lower concentration than ratjadon, preferably is (are) also to have a lower cytotoxicity than ratjadon and preferably is (are) to be easier to synthesise than ratjadon.

This aim is achieved by a formulation (termed “formulation according to the invention” below) for inhibition of the reproduction of tumor cells, comprising

(a) a substance of the formula II according to the invention or a mixture of two or more different substances of the formula II according to the invention in a sufficient concentration to inhibit the reproduction of tumor cells, and

(b) a pharmaceutically acceptable excipient.

In this context, pharmaceutically acceptable excipients are those excipients that are compatible with the other constituents of the formulation and do not have any unjustifiable harmful effects on those cells that are not to be influenced by the formulation. Pharmaceutically acceptable excipients, such as, for example, water, PBS (phosphate buffered saline) solution, emulsions such as oilin-water emulsions or triglyceride emulsions, are known to those skilled in the art. The formulation can be administered as a liquid or in the form of tablets, coated tablets or capsules. A person skilled in the art is easily able to select a suitable pharmaceutically acceptable excipient corresponding to the desired mode of administration.

Such formulations are advantageously suitable for inhibiting the reproduction of tumor cells in cell culture or in the living organism. By treating a tumor cell culture or a tumor with a formulation according to the invention it is possible to reduce or completely arrest the growth of tumor cells or tumors that respond to the substances according to the invention.

A further aim was to indicate a method for the preparation of a medicament where the medicament is to be suitable for inhibition of the cell growth of tumor cells. Preferably the active substance or all the active substances of the medicament should have a lower cytotoxicity than ratjadon. Likewise, the synthesis of the active substance or of the individual active substances should preferably be less laborious than that of ratjadon.

The aim is achieved by use of a substance of the formula II according to the invention or of a formulation according to the invention for the preparation of a medicament for inhibition of the reproduction of tumor cells. Such a medicament realises the advantages associated with the use of the substances according to the invention described above or the formulations according to the invention.

A further aim was to indicate a method for inhibition of the reproduction of cells, in particular of tumor cells. Preferably the cell cycle of the cells treated in accordance with the method is to be arrested.

The aim is achieved by the use of a substance of the formula II according to the invention or of a formulation according to the invention for inhibition of the reproduction of cells, in particular for inhibition of the reproduction of tumor cells. The advantages described above are associated with the use of a substance of the formula II according to the invention or of a formulation according to the invention. For example, the medicament is regularly less cytotoxic than a medicament in which the substance according to the invention or the substances according to the invention has or have been replaced by ratjadon in the same concentration.

The aim is likewise achieved by a method for (optionally non-therapeutic) inhibition of the reproduction of tumor cells where the tumor cells are exposed to a reproduction-inhibiting dose of a substance of the formula II according to the invention or to a reproduction-inhibiting dose of a mixture of two or more different substances of the formula II according to the invention. Such a method can, for example, be carried out on tumor cells in a cell culture (in vitro) instead of on a tumor in a living organism (in vivo).

A further aim was to indicate a substance that can serve as structural unit for the modular synthesis of a compound of the formula II according to the invention.

The aim is achieved by a substance of the formula

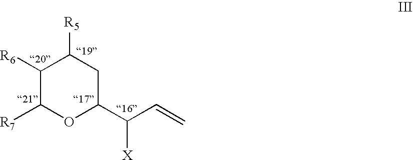

where X is an unprotected or protected hydroxyl group, R₅ is H, an unprotected or a protected hydroxyl group and R₆ and R₇ independently of one another are selected from the group that consists of H, CH₃, C₂H₅, n-C₃H₇, iso-C₃H₇,

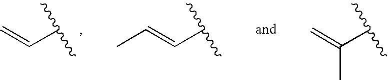

and where C“17” is R-configured if (a) C“16” is R-configured and at the same time (b) neither R₅ nor R₆ nor R₇ is H.

In this context a “protected hydroxyl group” is a side group that comprises a protective group such as, for example, TBS (SitBuMe₂) and that can be converted to an OH group by removal of this protective group.

For the sake of clarity the numbering of the carbon atoms has been so chosen that these correspond to the corresponding numbers in the end product. In addition the numbers have been placed in quotation marks.

Substances of the formula III with the indicated meanings for the radicals R₅ to R₇ and X are termed “fragments according to the invention” below.

Advantageously, the substances of the formula II according to the invention can be synthesized easily from such fragments according to the invention.

A fragment according to the invention in which R₅, R₆ and R₇ are each H is particularly preferred. Such substances can be prepared particularly easily and inexpensively. They can also be further processed particularly easily to give an end product according to formula II, in which R₅, R₆ and R₇ are each H.

A further aim was to indicate a method for the preparation of a substance of the formula II according to the invention.

The aim is achieved by a method in which a fragment according to the invention (cf. formula III), in which, in each case, X is a protected hydroxyl group and R₅ is H or a protected hydroxyl group, is linked to an iodide of the formula

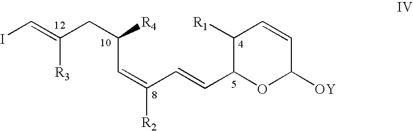

in which R₁, R₂ and R₃ independently of one another are selected from the group that consists of H, CH₃ and C₂H₅, R4 is CH₃ or C₂H₅ and Y is methyl, ethyl or isopropyl.

Linkage (Heck coupling) of a fragment according to the invention with an iodide of the formula IV is particularly advantageous since in this way it is possible to dispense with the use of pharmacologically undesired tin compounds.

In the examples below the synthesis of a substance 1 of the formula II according to the invention and of a fragment A of the formula III according to the invention is first described. The action of a few preferred substances 1, 36 and 37 of the formula II according to the invention is then described in more detail. Before this, however, a few general remarks with regard to the descriptions of the experiments:

¹H NMR spectra were recorded using the Bruker WP-200 SY, AM-400 and AM-500 instruments. Unless indicated otherwise, tetramethylsilane (TMS) served as internal standard. Unless indicated otherwise, deuterochloroform (CDCl3) was used as solvent. The chemical shifts are indicated in ppm on the 6 scale. The coupling constants are given in Hertz (Hz). The signal multiplicities are characterised as follows:

s=singlet, d=doublet, t=triplet, q=quartet, qui=quintet, m=multiplet, dd=double doublet, dt=double triplet, dq=double quartet, br=broad.

¹³C NMR spectra were recorded using the abovementioned instruments at 100, 200 or 250 MHz with TMS as internal standard. Unless indicated otherwise, CDCl3 served as solvent. The spectra were recorded using the APT- or DEPT-method.

Infrared spectra (1R) were recorded either in CHCl₃ using the Perkin-Elmer 580 electrophotometer, as KBr compact or as capillary film using the Perkin-Elmer 1710 FT spectrophotometer; in addition the Bruker IFS 25 and Vector-22 instruments were used for IR measurements. The characteristic bands are indicated in wave numbers ν [cm⁻¹].

Mass spectra (MS, MS-FAB, HRMS) were recorded using the Finnigan MAT 312 or VG Autospec instruments at an ionisation potential of 70 eV. The m/z ratios are indicated in each case, the signal intensities being indicated in % of the base peak.

Angles of rotation [α] were measured using the Perkin Elmer 341 polarimeter. The wavelengths used, the temperature, the solvent and the concentration (in 10 mg/ml) of the substance for measurement are indicated.

Elemental analyses (EA) were carried out using the Heraus CHN-Rapid instrument.

Melting points were determined using a Buchi apparatus according to Dr. Tottoli and not corrected.

Bulb tube distillations were carried out using a Buchi GKR 50 bulb tube oven; the indicated temperatures relate to the air bath.

Gas chromatograms were recorded using a Hewlett-Packard HP 6890-II with a SE-54 capillary column (25 m, Macherey-Nagel) and flame ioniser, nitrogen serving as carrier gas. Chiral gas chromatograms were recorded using a HewlettPackard HP 5890-II and a chiral column (Lipodex E No. 723368, Octacis-(2,6-di-O-pentyl-O-butyryl)-γ-cyclodextrin as stationary phase) from Macherey-Nagel.

Column chromatography was carried out using silica gel (particle size 40-60 μm, pore diameter 60 Å) from J. T. Baker under slight excess pressure.

Analytical thin layer chromatography was carried out on Merck 60F₂₅₄ silica gel-coated aluminium foils (coating thickness 0.2 mm). The colouring reagents used were vanilla, cerium, bromocresol green or DNPH solutions.

Solvents were employed only in the distilled form. Absolute solvents were dried in accordance with the known methods (Perrin, D. D.; Armarego, W. L. F. Purification of Laboratory Chemicals, 3rd Ed., Pergamon Press Oxford, 1988) and stored over molecular sieve, CaH₂ or Na. THF was distilled over sodium/benzophenone in a nitrogen atmosphere and Et₂O over sodium in an argon atmosphere.

R actions were carried out under an argon atmosphere. Unless indicated otherwise, a magnetic stirrer was used in all experiments.

### Example 1

### General Synthesis Scheme for Substances of the Formula II According to the Invention

The synthesis is carried out in modular fashion from three fragments of the formulae

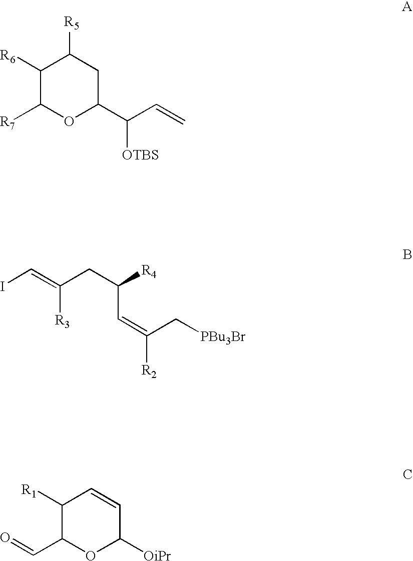

with the meanings described above for the radicals R₁ to R₇.

For synthesis of an A fragment (see Fig. Error! Reference source not found.) an aldol adduct 2 is transamidated to give the Weinreb amide 3. The Weinreb amide 3 is then TBS-protected to give the amide 4. The amide 4 is reduced to give the aldehyde 5. Aldehyde 5 is reacted with the ketene acetal 6 in a vinylogenous Mukaiyama aldol reaction to give the hydroxy ester 7. Hydroxy ester 7 is converted to the allyl alcohol 8. Allyl alcohol 8 is epoxidised to give the epoxide 9. Epoxide 9 is deprotected to give the epoxytriol 10. Epoxytriol 10 is cyclised to give the tetrahydropyran-triol 11. Tetrahydropyran-triol 11 is protected to give the tris-TBS ether 12. The primary alcohol group of compound 12 is deprotected to give the alcohol 13. Alcohol 13 is oxidised to give the aldehyde 14. Finally, aldehyde 14 is converted to an olefin to give an A fragment Al.

For synthesis of a B fragment (see Fig. Error! Reference source not found.) the alkyne 15 is carbometallated to give the alcohol 16. The alcohol 16 is oxidised to give the aldehyde 17. Aldehyde 17 is converted to the ester 18 in a Still-Gennari olefination. Ester 18 is reduced to give the alcohol 19. Alcohol 19 is brominated to give the bromide 20. Bromide 20 is converted to the phosphonium salt B1.

The synthesis of a C fragment (see Fig. Error! Referenc source not found.) starts with a hetero-Diels-Alder reaction to give the ester 21. Ester 21 is reduced to give the alcohol 22. Alcohol 22 is oxidised to give the aldehyde Cl.

For synthesis of the substance 1 according to the invention (see Fig. Error! Reference source not found.) the fragments B1 and C1 are linked to one another in a Wittig reaction to give compound 23. Compound 23 and fragment A1 are reacted in a Heck coupling to give compound 24. Compound 24 is oxidised to give the lactone 25. Lactone 25 is converted into the ratjadon derivative 1 by deprotection.

The synthesis of a preferred A fragment, i.e. the fragment A1, is described in more detail in Examples 2 to 13 below.

The synthesis of a B fragment, i.e. the fragment B1, is described in more detail in Examples 14 to 19.

The synthesis of a C fragment, i.e. the fragment C1, is described in more detail in Examples 20 to 22.

The synthesis of the preferred substance 1 from the fragments A1, B1, and C1 is described in more detail in Examples 23 to 26.

The synthesis of the preferred compound 35, an alternative A fragment, is described in more detail in Examples 27 to 35. This compound differs structurally from the A1 fragment described above in that its tetrahydropyran ring is only H-substituted at the positions C“19”, C“20” and C“21” (in this context see formula III) (the radicals R₅, R₆ and R₇ according to formula III are thus H).

Compared with the synthesis of the A1 fragment described in Examples 2 to 13, the synthesis of the preferred compound 35 is advantageously simplified. Compound 35 can be used in exactly the same way as the described A1 fragment in Examples 23 to 26 for the synthesis of a preferred substance of the formula II, i.e. the compound 36.

For synthesis of the preferred compound 35 (see Fig. Error! Bookmark not defined.) an aldehyde 27 is synthesized from the diol 26. Aldehyde 27 is converted to the ester 28 in a Wittig-Horner reaction. Ester 28 is reduced to give the allyl alcohol 29. Allyl alcohol 29 is converted to the epoxide 30 in an asymmetric Sharpless epoxidation. Epoxide 30 is cyclised to give the diol 31. Diol 31 is double protected to give the TBS ether 32. The primary OH group of the TBS ether 32 is deprotected to give the mono-protected alcohol 33. Alcohol 33 is converted to the aldehyde 34 in a Dess-Martin oxidation. Aldehyde 34 is converted to the preferred olefin 35 in a Tebbe olefination.

### Example 2

### Transamidation of the Aldol Adduct 2 to Give the Weinreb Amide 3

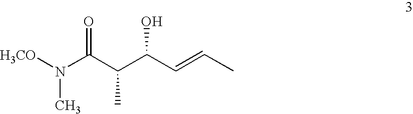

Trimethylaluminium (70 ml, 140 mmol, 2M in toluene) is added dropwise to a suspension (0° C.) of N,O-dimethylhydroxylamine hydrochloride (13.57 g, 139 mmol) in 200 ml CH₂Cl₂ over a period of 40 min. The solution is heated to room temperature and stirred for 1 h at this temperature. It is then cooled to −20° C. and a solution of the aldol adduct 2 (20 g, 66 mmol, described in D. A. Evans et al., J. Org. Chem. 1990, 55, 6260-6268) in 50 ml CH₂Cl₂ is then added using a syringe. The mixture, which has become turbid, is heated to room temperature over a period of 5 h and then stirred overnight. The solution is transferred by a syringe into 400 ml aqueous tartaric acid (1M) (0° C.) and this mixture is stirred vigorously for 1 h. The phases are separated and the aqueous phase is extracted with CH₂Cl₂ (3×100 ml). The combined organic phases are washed with saturated aqueous NaCl solution, dried with MgSO₄ and concentrated under vacuum. The crude product can be used in the next reaction without further purification. By means of column chromatography (EtOAc/petroleum ether 1:1) amide 3 is obtained as a colourless oil: R_{f}=0.20 (EtOAc/petroleum ether 1:1); [α]²⁰_{D}=−24.8 (c 1.1, CHCl₃); ¹ H-NMR (400 MHz, CDCl₃) δ 5.71 (ddq, J=14.3, 6.5, 1.3 Hz, 1H), 5.44 (ddq, J=14.3, 6.3, 1.6 Hz, 1H), 4.31 (m, 1H), 3.66 (s, 3H), 3.47 (bs, 1H), 3.15 (s, 3H), 2.89 (bs, 1H), 1.66 (ddd, J=6.5, 1.6, 1.0 Hz, 3H), 1.13 (d, J=7.2 Hz, 3H); ¹³C-NMR (100 MHz, CDCl₃) δ 177.7, 130.6, 127.5, 72.5, 61.5, 39.7, 31.8, 17.7, 10.7; IR 3415, 1637 cm⁻¹; MS (EI) m/z (%)=187 (5) [M]⁺, 127 (25), 117 (35), 71 (100); HRMS calculated for C₉H₁₇NO₃: 187.1208, found 187.1208.

### Example 3

### TBS Protection of the Weinreb Amide 3 to Give the Amide 4

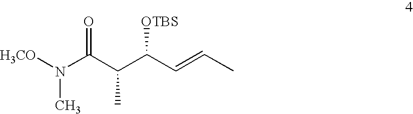

2,6-Lutidine (13.5 ml, 116 mmol) and TBSSOTf (20 ml, 87 mmol) are added successively to a solution (0° C.) of the non-purified alcohol 3 in 300 ml CH₂Cl₂. The reaction [lacuna] is stirred for 15 min at 0° C. and then heated to room temperature. The excess triflate is quenched by the addition of 2.5 ml methanol. The solution is diluted with 300 ml CH₂Cl₂ and washed with saturated aqueous NaHCO₃ solution (2×200 ml). The aqueous phases are extracted with CH₂Cl₂ (100 ml) and the combined organic phases are washed [lacuna] 1M aqueous NaHSO₄ solution (3×200 ml). The organic phases are washed with saturated aqueous NaCl solution, dried with MgSO₄ and concentrated under vacuum. The crude product can then be used directly in the next reaction. By means of a separation by column chromatography (petroleum ether/EtOAc 2:1) amide 4 is obtained as a colourless oil: R_{f}=0.50 (EtOAc/petroleum ether 1:1); [α]20D=+0.7 (c 1.0, CHCl₃); ¹ H-NMR (400 MHz, CDCl₃) δ 5.54 (ddq, J=15.4, 6.4, 0.9 Hz, 1H), 5.38 (ddq, J=15.4, 7.3, 1.0 Hz, 1H), 4.12 (m, 1H), 3.62 (s, 3H), 3.10 (s, 3H), 2.95 (bs, 1H), 1.60 (ddd, J=6.4, 1.6, 0.5 Hz, 3H), 1.14 (d, J=6.8 Hz, 3H), 0.86 (s, 9H), 0.02 (s, 3H), −0.02 (s, 3H); ¹³C-NMR (100 MHz, CDCl₃) δ 176.0, 133.0, 126.6, 75.7, 61.4, 42.8, 32.0, 25.9, 18.2, 17.5, 14.5, −4.1, −4.8; IR 1663 cm⁻¹; MS (EI) m/z (%)=302 (4) [M]⁺, 244 (82), 185 (73), 142 (42), 115 (34), 89 (69), 73 (100); HRMS calculated for C₁₅H₃₁NO₃Si: 301.2073, found 301.2075.

### Example 4

### Reduction of the Amide 4 to Give the Aldehyde 5

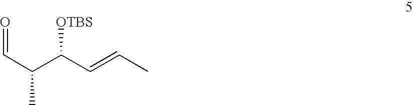

Diisobutylaluminium hydride (140 ml, 168 mmol, 1.2 M in toluene) is added dropwise over a period of 1 h to a solution (−78° C.) of the crude product 4 (≈66 mmol) from the preceding reaction in 400 ml THF and the resulting solution is stirred for a further 15 min at this temperature. The excess diisobutylaluminium hydride is quenched by the addition of 8 ml acetone. The solution is transferred using a syringe into a vigorously stirred mixture of 600 ml 1 M aqueous tartaric acid and 500 ml petroleum ether. After 1 h ether (800 ml) is added, the phases are separated and the aqueous phase is extracted with ether (2×300 ml). The combined organic phases are washed with saturated aqueous NaCl solution, dried with MgSO₄ and concentrated under vacuum. After purification by flash chromatography (petroleum ether/EtOAc 20:1) the aldehyde 5 (13.3 g, 83%) is obtained as a colourless oil.

R_{f}=0.56 (petroleum ether/EtOAc 5:1); [A]²⁰_{D}=−43.0 (c 1.0, CHCl₃); ¹H-NMR (400 MHz, CDCl₃) δ 9.74 (d, J=1.4 Hz, 1H), 5.62 (ddq, J=15.3, 6.5, 1.0 Hz, 1H), 5.41 (ddq, J=15.2, 7.2, 1.6 Hz, 1H), 4.41 (ddt, J=7.2, 4.7, 0.9 Hz, 1H), 2.43 (ddq, J=6.9, 4.7, 1.4 Hz, 1H), 1.67 (ddd, J=6.4, 1.6, 0.8 Hz, 3H), 1.02 (d, J=6.9 Hz, 3H), 0.84 (s, 9H), 0.02 (s, 3H), −0.01 (s, 3H); ¹³C-NMR (100 MHz, CDCl₃) δ 205.0, 131.3, 127.5, 73.8, 52.9, 25.7, 18.1, 17.5, 8.6, −4.1, −5.0; IR (CHCl₃) 1721 cm⁻¹; MS (EI) m/z (%)=227 (1) [M−CH₃]⁺, 201 (49), 185 (61), 75 (100); HRMS calculated for C₁₂H₂₃O₂Si: 227.1467, found 227.1471.

### Example 5

### Vinylogous Mukaivama Aldol Reaction of Aldehyde 5 to Give the Hydroxy Ester 7

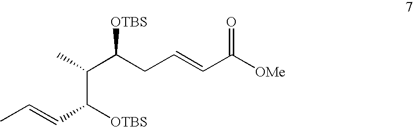

Tris(pentafluorophenyl)borane (1.02 g, 2 mmol) is added to a solution (−78° C.) of the aldehyde 5 (2.42 g, 10 mmol) and the ketene acetal 6 (4.29 g, 20 mmol) in 100 ml CH₂Cl₂/Et₂O (9:1). The solution is heated to room temperature and concentrated under vacuum. The solid residue is purified by flash chromatography (petroleum ether/EtOAc 18:1). Hydroxy ester 7 (3.38 g, 74%) is obtained as a colourless oil: R_{f}=0.59 (petroleum ether/EtOAc 5:1); [α]²⁰_{D}=+3.8 (c 0.5, CHCl₃); ¹H-NMR (400 MHz, CDCl₃) δ 6.89 (dt, J=15.7, 7.5 Hz, 1H), 5.81 (dt, J=15.7, 1.5 Hz, 1H), 5.49 (ddq, J=15.3, 6.3, 0.6 Hz, 1H), 5.36 (ddq, J=15.3, 7.8, 1.1 Hz, 1H), 4.04 (dd, J=7.8, 5.9 Hz, 1H), 3.79 (dt, J=5.9, 4.4 Hz, 1H), 3.71 (s, 3H), 2.40 (m, 2H), 1.65 (dd, J=6.4, 1.6 Hz, 3H), 1.49 (ddq, J=6.9, 5.9, 4.4 Hz, 1H), 0.87 (d, J=6.9 Hz, 3H), 0.84-0.87 (2s, 9H), −0.03-0.02 (4s, 12H); ¹³C-NMR (100 MHz, CDCl₃) δ 168.8, 146.5, 133.8, 126.5, 122.7, 74.6, 71.7, 51.4, 44.6, 38.2, 25.9 (2C), 18.12 (2C), 17.6, 9.7, −3.7, −3.9, −4.5, −4.8; IR (CHCl₃) 1716 cm⁻¹; MS (EI) m/z (%)=441 (1) [M−CH₃]⁺, 399 (29) [M-tBu]⁺, 317 (35), 243 (46), 185 (100), 147 (24), 73 (71); HRMS calculated for C₂₃H₄₅O₄Si₂: 441.2856 found 441.2855.

The other diastereomer at the newly generated centre of asymmetry can be prepared by using BF₃ instead of tris(pentafluorophenyl)borane.

### Example 6

### Reduction of the Hydroxy Ester 7 to Give the Allyl Alcohol 8

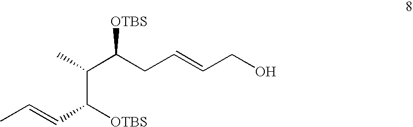

Diisobutylaluminium hydride (33 ml, 40 mmol, 1.2 M in toluene) is added to a solution (−78° C.) of the hydroxy ester 7 (6 g, 13 mmol) in 100 ml CH₂Cl₂. The solution is stirred for 1 h at this temperature, diluted with 100 ml MTBE and heated to room temperature. After adding 3.3 ml H₂O, the mixture is stirred vigorously until a white gel has formed. NaOH (3.3 ml, 4M) and H₂O (6.6 ml) are added to this gel and the suspension is stirred until a white solid has formed. The mixture is dried with MgSO₄ and the solids are separated off by filtration. The filtrate is concentrated under vacuum and purified by flash chromatography (petroleum ether/EtOAc 6:1). Allyl alcohol 8 (5.58 g, 99%) is obtained as a colourless oil: R_{f}=0.41 (petroleum ether/EtOAc 5:1); ¹ H-NMR (400 MHz, CDCl₃) 85.62 (m, 2H), 5.49 (ddq, J=15.4, 6.3, 0.6 Hz, 1H), 5.35 (ddq, J=15.3, 7.8, 1.5 Hz, 1H), 4.06 (m, 2H), 4.01 (m, 1H), 3.72 (ddd, J=6.8, 6.4, 4.0 Hz, 1H), 2.25 (m, 2H), 1.65 (ddd, J=6.4, 1.5, 0.5 Hz, 3H), 1.49 (ddq, J=6.8, 6.4, 4.0 Hz, 1H), 0.87 (s, 9H), 0.86 (d, J=6.8 Hz, 3H), 0.85 (s, 9H), 0.01 (s, 3H), 0.00 (s, 3H), −0.01 (s, 3H), −0.04 (s, 3H); ¹³CNMR (100 MHz, CDCl₃) δ 134.1, 131.1, 129.6, 126.3, 74.9, 72.1, 63.8, 43.9, 38.2, 26.0 (2C), 18.2 (2C), 17.6, 9.4, −3.7, −3.8, −4.5, −4.7; IR (CHCl₃): ν=3613 cm⁻¹; MS (EI) m/z (%)=289 (3), 259 (3), 235 (1), 225 (1), 215 (2), 185 (100), 145 (14), 75 (13), 73 (26); HRMS calculated for C₁₁H₂₃O₂Si: 215.1467, found 215.1467.

### Example 7

### Epoxidation of the Allyl Alcohl (sic) 8 to Give the Epoxide 9

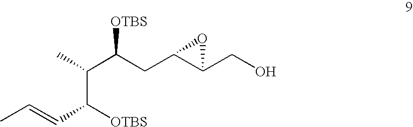

NaHCO₃ (0.92 g, 10.9 mmol) is added to a solution of the allyl alcohol 8 (2.44 g, 5.7 mmol) in 75 ml CH₂Cl₂ at 0° C. After adding 70% mCPBA (1.53 g, 6.2 mmol) the suspension is stirred for 3 h at 0° C. and quenched by adding saturated aqueous NaHCO₃ solution (50 ml). The aqueous phase is extracted with CH₂Cl₂ (2×50 ml) and the combined organic phases are washed successively with 2N NaOH, H₂O and saturated NaCl solution. The organic phase is dried with MgSO₄ and concentrated under vacuum. After purification by flash chromatography (petroleum ether/EtOAc 8:1) the epoxide 9 (2.21 g, 87%) is obtained as a colourless oil: R_{f}=0.27 (petroleum ether/EtOAc 5:1); [α]²⁰_{D}=+17.8 (c 1.0, CHCl₃); ¹H-NMR (400 MHz, CDCl₃) δ 5.50 (ddq, J=15.4, 6.2, 0.6 Hz, 1H), 5.39 (ddq, J=15.3, 7.8, 1.3 Hz, 1H), 4.10 (dd, J=7.7, 5.4 Hz, 1H), 3.89 (dd, J=12.3, 2.2 Hz, 1H), 3.84 (ddd, J=6.9, 5.7, 4.1 Hz, 1H), 3.57 (dd, J=12.3, 4:4 Hz, 1H), 2.98 (ddd, J=7.0, 4.7, 2.4 Hz, 1H), 2.89 (m, 1H), 1.80 (ddd, J=14.3, 6.9, 4.7 Hz, 1H), 1.65 (ddd, J=6.2, 1.4, 0.6 Hz, 3H), 1.64 (ddd, J=14.3, 7.0, 5.6 Hz, 1H), 1.59 (ddq, J=6.9, 5.4, 4.1 Hz, 1H), 0.87 (s, 9H), 0.87 (d, J=7.0 Hz, 3H), 0.84 (s, 9H), −0.05-0.03 (4s, 12H); ¹³C-NMR (100 MHz, CDCl₃) δ 134.2, 126.2, 74.1, 71.1, 61.6, 58.7, 53.4, 45.2, 36.9, 25.9 (2C), 25.9, 18.1 (2C), 17.6, 9.8, −3.6, −4.2, 4.4, 4.7; IR (CHCl₃) 3402 cm⁻¹; MS (EI) m/z (%)=305 (4), 287 (2), 259 (2), 227 (2), 185 (100), 147 (15), 75 (18), 73 (27); HRMS calculated for C₁₀H₂₁OSi: 185.1362, found 185.1361.

The other diastereomer can be prepared by using the analogous compound with Z double bond.

### Example 8

### Deprotection of the Epoxide 9 to Give the Epoxytriol 10

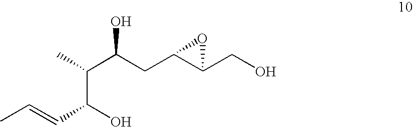

TBAF (20 ml, 20 mmol, 1.0 M in THF) is added to a solution of the epoxide 9 (3.03 g, 6.8 mmol) in 100 ml THF and the solution is stirred at room temperature for 48 h. The reaction is quenched by adding saturated aqueous NH₄Cl solution (50 ml). The phases are separated and the aqueous phase is extracted with EtOAc (6×50 ml). The combined organic phases are dried (MgSO₄) and concentrated under vacuum. The crude product is filtered through a short silica gel column with EtOAc. The epoxytriol 10, which has already been partially cyclised to the tetrahydropyran-triol 11, is obtained as a colourless oil. The product can be used directly in the next reaction or can be purified by flash chromatography (EtOAc/MeOH 15:1): ¹H NMR (400 MHz, CDCl₃) δ 5.67 (ddq, J=15.3, 1.3, 6.4 Hz, 1H), 5.53 (ddq, J=15.3, 6.0, 1.5 Hz, 1H), 4.09 (m, 1H), 4.33 (m, 1H), 3.61 (m, 1H, 3.82 (m, 1H), 3.38 (bs, 1H), 3.08 (m, 1H), 2.97 (m, 1H), 2.66 (bs, 1H), 2.38 (bs, 1H), 1.92 (ddd, J=14.0, 9.7, 4.1 Hz, 1H), 1.70 (dt, J=6.3, 1.3 Hz, 3H), 1.55 (ddq, J=3.0, 2.3, 6.9 Hz, 1H), 1.46 (ddd, J=14.3, 6.9, 3.6 Hz, 1H), 0.90 (d, J=7.1 Hz, 3H); ¹³C-NMR (100 MHz, CDCl₃) δ 133.0, 126.9, 77.5, 73.4, 62.2, 58.9, 54.3, 42.9, 37.4, 17.8, 5.6; IR 3345, 966 cm⁻¹; HRMS calculated for C₁₁H₂₀O₄: 213.1362, found 213.1361.

### Example 9

### Cyclisation of the Epoxytriol 10 to Give the Tetrahydropyran-Triol 11

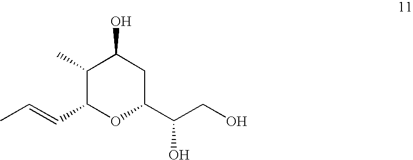

Amberlyst 15 (40 mg) is added to a solution of the epoxytriol 10 (6.8 mmol) in 50 ml THF. After 6 h the resin is filtered off and the filtrate is concentrated under vacuum. After purification by flash chromatography (EtOAc/MeOH 15:1) the tetrahydropyran-triol 11 (1.21 g, 82%) is obtained as a colourless oil: [α]²⁰_{D}=−2.0 (c 1.0, CHCl₃); ¹ H-NMR (400 MHz, CDCl₃) δ 5.64 (ddq, J=15.4, 1.4, 6.5 Hz, 1H), 5.40 (ddq, J=15.4, 6.0, 1.6 Hz, 1H), 4.39 (m, 1H), 3.99 (q, J=2.9 Hz, H) 3.94 (ddd, J=12.3, 4.4, 1.5 Hz, 1H), 3.61-3.78 (m, 3H), 1.78 (ddd, J=14.1, 12.3, 2.9 Hz, 1H), 1.78 (dt, J=6.5, 1.4 Hz, 3H), 1.68 (m, 1H), 1.50 (m, 1H), 0.89 (d, J=7.2 Hz, 3H); ¹³C-NMR (100 MHz, CDCl₃) δ 129.9, 126.8, 74.8, 74.2, 73.5, 70.0, 63.5, 39.6, 29.1, 17.9, 11.2; IR 3682, 3609, 3470 cm⁻¹; HRMS calculated for C₁₁H₂₂O₄: 216.1362, found 216.1364.

### Example 10

### Protection of the Tetrahydropyran-Triol 11 to Give the Tris-TBS Ether 12

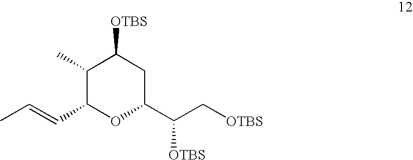

2,6-Lutidine (3.2 ml, 27.5 mmol) and TBSOTf (4.8 ml, 20.9 mmol) are added successively to a solution (−78° C.) of the tetrahydropyran-triol 11 (1 g, 4.6 mmol) in 100 ml CH₂Cl₂. The solution is heated to room temperature and quenched by adding saturated aqueous NaHCO₃ solution (50 ml). The phases are separated and the aqueous phase is extracted with CH₂Cl₂ (2×50 ml). The combined organic phases are washed first with 1M aqueous NaHSO₄ solution (3×50 ml) and then with saturated aqueous NaCl solution, dried with MgSO₄ and concentrated under vacuum. After purification by flash chromatography (petroleum ether/EtOAc 40:1) the TBS ether 12 (2.25 g, 87%) is obtained as a colourless oil: R_{f}=0.17 (petroleum ether/EtOAc 50:1); [α]²⁰_{D}=−23.8 (c 1.0, CHCl₃); ¹ H-NMR (400 MHz, CDCl₃) δ 5.61 (ddq, J=15.4, 6.4, 1.5 Hz, 1H), 5.37 (ddq, J=15.4, 5.6, 1.6 Hz, 1H), 4.37 (m, 1H), 3.86 (m, 2H), 3.72 (m, 1H), 3.49 (m, 2H), 1.72 (ddd, J=13.8, 11.8, 2.6 Hz, 1H), 1.67 (dt, J=6.5 Hz, 3H), 1.47 (m, 1H), 1.34 (ddd, J=13.8, 2.5, 1.3 Hz, 1H), 0.87-0.86 (3s, 27H), 0.83 (d, J=7.2 Hz, 3H), 0.07-0.00 (4s, 18H); ¹³C-NMR (100 MHz, CDCl₃) 6 131.3, 125.2, 75.8, 74.5, 72.5, 70.9, 64.5, 40.4, 28.1, 26.0, 25.9, 25.8, 18.3, 18.2, 18.1, 17.9, 11.2, −4.4, −4.5, −4.8, −5.0, −5.5 (2C); MS (EI) m/z (%)=558(4) [M]⁺, 501(25) [M-tBu]⁺, 419(30), 369(17), 327(18) 287(39), 261(36), 227(53), 171(69), 147(34), 73(100); HRMS calculated for C₂₉H₆₂O₄Si₃: 558.3956 found 558.3956.

### Example 11

### Selective Deprotection of the Primary Alcohol Group of Compound 12 to Give the Alcohol 13

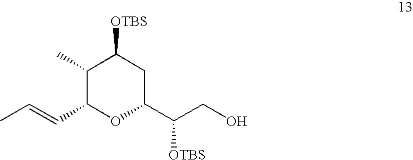

Chloroform (100 ml) and concentrated aqueous HCl (20 ml) are mixed in a separating funnel and the organic phase is separated off. The TBS ether 12 from the previous reaction (2.6 g, 4.7 mmol) is dissolved in the organic phase and the solution thus obtained is stirred for 4 h at room temperature. The reaction is quenched by adding saturated aqueous NaHCO₃ solution (50 ml). The phases are separated and the aqueous phase is extracted with CHCl₃ (2×50 ml). The combined organic phases are dried with MgSO₄ filtered and concentrated under vacuum. After purification by flash chromatography (petroleum ether/EtOAc 15:1) the alcohol 13 (2.01 g, 97%) is obtained as a colourless oil: R_{f}=0.34 (petroleum ether ((sic) EtOAc 10:1); [α]²⁰_{D}=−14.3 (c 1.0, CHCl₃) ¹ H-NMR (400 MHz, CDCl₃) δ 5.58 (ddq, J=15.4, 6.4, 1.4 Hz, 1H), 5.37 (ddq, J=15.4, 5.9, 1.6 Hz, 1H), 4.38 (m, 1H), 3.85 (q, J=2.9 Hz, 1H), 3.80 (m, 1H), 3.65-3.55 (m, 3H), 2.60 (s, 1H), 1.66 (d, J=6.4 Hz, 3H), 1.62-1.47 (m, 3H), 0.87 (2s, 18H), 0.84 (d, J=7.2 Hz, 3H), 0.07 (2s, 6H), 0.02 (4s, 6H); ¹³C-NMR (100 MHz, CDCl₃) δ 130.5, 126.0, 75.4, 74.9, 74.3, 70.5, 65.7, 40.2, 31.1, 25.8 (2C), 18.1, 18.0, 17.9, 11.2, −4.4, −4.6, −4.9 (2C); IR 3460 cm⁻¹; MS (EI) m/z (%)=444 (6) [M]⁺, 387 (21) [M-tBu]⁺, 287 (66), 255 (73), 219 (60) 173 (100), 171 (79); HRMS calculated for C₂₃H₄₈O₄Si₂: 444.3091, found 444.3091.

### Example 12

### Oxidation of the Alcohol 13 to Give the Aldehyde 14

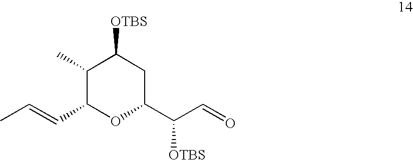

Dess-Martin periodinane (800 mg, 1.89 mmol) is added to a solution (0° C.) of the alcohol 13 (700 mg, 1.57 mmol) in 50 ml CH₂Cl₂. The solution is heated to room temperature and stirred for a further 3 h. The reaction is quenched by adding a solution of Na₂S₂O₃ 5H₂O (2.5 g) in saturated aqueous NaHCO₃ solution (25 ml) and [lacuna] stirred vigorously until a clear solution has formed. The aqueous phase is extracted with CH₂Cl₂ (2×25 ml). The combined organic phases are dried (MgSO₄) and concentrated under vacuum. After purification by flash chromatography (petroleum ether/EtOAc 15:1) the aldehyde 14 (641 mg, 92%) is obtained as a colourless oil: R_{f}=0.64 (petroleum ether/EtOAc 10:1); [α]²⁰_{D}=−19.4 (c 1.0, CHCl₃); ¹ H-NMR (400 MHz, CDCl₃) 59.61 (d, J=1.3 Hz, 1H), 5.60 (ddq, J=15.4, 6.5, 1.5 Hz, 1H), 5.36 (ddq, J=15.4, 5.6, 1.6 Hz, 1H), 4.39 (m, 1H), 4.08 (ddd, J=11.3, 4.6, 2.2 Hz, 1H), 4.04 (dd, J=4.6, 1.4 Hz, 1H), 3.86 (q, J=2.9 Hz, 1H), 1.80 (ddd, J=13.7, 11.4, 2.2 Hz, 1H), 1.67 (d, J=6.7 Hz, 3H), 1.50 (m, 1H), 1.30 (ddd, J=13.7, 2.2, 1.4 Hz, 1H), 0.90 (s, 9H), 0.86 (s, 9H), 0.83 (d, J=7.2 Hz, 3H), 0.07 (2s, 6H), 0.01 (s, 3H), 0.00 (s, 3H); ¹³C-NMR (100 MHz, CDCl₃) δ 203.7, 130.5, 125.8, 80.4, 74.8, 73.3, 70.4, 40.1, 29.3, 25.7 (2C), 18.3, 18.0, 17.9, 11.1, 4.8 (2C), 4.9 (2C); IR 1738 cm⁻¹; MS (EI) m/z (%)=442 (3) [M]⁺, 385 (14) [M-tBu]⁺, 303 (55), 187 (90), 73 (100); HRMS calculated for C₁₉H₃₇O₄Si₂: 385.2230, found 385.2231.

### Example 13

### Olefination of the Aldehyde 14 to Give the A1 Fragment

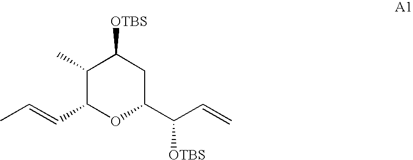

The Tebbe reagent (3.2 ml, 1.60 mmol, 0.5 M in toluene) is added to a solution (0° C.) of the aldehyde 14 from the previous reaction (700 mg, 1.58 mmol) in 50 ml THF. After 15 min at this temperature the solution is diluted with 50 ml Et₂O and quenched by slowing adding 0.6 ml 1 M NaOH. The mixture thus obtained is dried with MgSO₄, filtered and the filtrate is concentrated under vacuum. After separation by flash chromatography, the A1 fragment (662 mg, 95%) is obtained as a colourless oil: R_{f}=0.68 (petroleum ether/EtOAc 20:1); [α]²⁰_{D}=−11.3 (c 1.0, CHCl₃); ¹ H-NMR (400 MHz, CDCl₃) δ 5.79 (ddd, J=17.2, 10.5, 5.0 Hz, 1H), 5.63 (ddq, J=15.4, 6.5, 1.5, 1H), 5.39 (ddq, J=15.4, 5.8, 1.6 Hz, 1H), 5.23 (d, J=17.2 Hz, 1H), 5.07 (d, J=10.5 Hz, 1H), 4.38 (m, 1H), 4.23 (m, 1H), 3.85 (q, J=2.9 Hz, 1H), 3.67 (ddd, J=11.7, 3.9, 2.3 Hz, 1H), 1.73 (ddd, J=13.8, 11.7, 2.3 Hz, 1H), 1.67 (d, J=6.7 Hz, 3H), 1.47 (m, 1H), 1.30 (ddd, J=13.8, 2.3, 1.3 Hz, 1H, 0.89 (s, 9H), 0.86 (s, 9H), 0.83 (d, J=7.2 Hz, 3H), 0.07-0.00 (4s, 12H); ¹³C-NMR (100 MHz, CDCl₃) δ 138.7, 131.2, 125.3, 114.6, 75.7, 75.6, 74.6, 70.9, 40.3, 27.5, 25.9, 25.8, 18.3, 18.0, 17.9, 11.1, −4.6 (2C), −4.8, −4.9; MS (EI) m/z (%)=40 (5) [M]⁺, 383 (15), 301 (69), 187 (52), 143 (100); HRMS calculated for C₂₄H₄₈O₃Si₂: 440.3142, found 440.3141.

### Example 14

### Carbometallation of the Alkyne 15 to Give the Alcohol 16

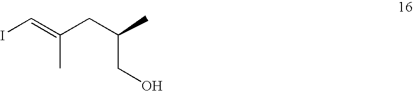

AlMe₃ (15.30 ml, 2 M in toluene) is added slowly to a solution (−10° C.) of Cp₂ZrCI₂ (1.20 g, 4.09 mmol) in 7 ml CH₂Cl₂. After 10 min the alkyne 15 (1.00 g, 10.19 mmol) in 10 ml CH₂Cl₂ is added dropwise and the solution is stirred for 12 h. The solution is cooled to −40° C. and 12 (2.85 g, 11.23 mmol), dissolved in 12 ml THF, is added dropwise. The solution is stirred for 1 h and then quenched with saturated aqueous NaHCO₃ solution at −20° C. The phases are separated and the aqueous phase is extracted with MTBE. The combined organic phases are dried with MgSO₄, filtered and concentrated under vacuum. After purification by flash chromatography (petroleum ether/EtOAc 2:1), the alcohol 16 (2.03 g, 83%) is obtained as a colourless liquid: R_{f}=0.30 (petroleum ether/EtOAc 2:1); [α]²⁰D+9.4 (c 1.0, CHCl₃); ¹H-NMR (400 MHz, CDCl₃) δ 5.87 (d, J=0.8 Hz, 1H), 3.40-3.45 (m, 2H), 2.33 (dd, J=13.4, 6.0 Hz, 1H), 1.99 (dd, J=13.4, 8.4 Hz, 1H), 1.80-1.85 (m 1H), 1.80 (d, J=0.98 Hz, 3H), 0.85 (d, J=6.8 Hz, 3H); ¹³C-NMR (100 MHz, CDCl₃) δ 146.5, 75.6, 67.6, 43.6, 33.7, 23.7, 16.3; IR 3302, 2870, 1456, 1377 cm⁻¹; HRMS calculated for C₇H₁₃IO: 240.0011; found 240.0011.

### Example 15

### Oxidation of the Alcohol 16 to Give the Aldehyde 17

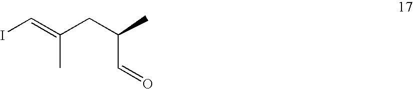

Dess-Martin periodinane (3.99 g, 9.41 mmol) is added to a solution (0° C.) of the alcohol 16 (1.75 g, 7.29 mmol) in 60 ml CH₂Cl₂. The solution is heated to room temperature and stirred for a further 3 h. The reaction is quenched by adding a solution of Na₂S₂O₃ 5H₂O (2.5 g) in saturated aqueous NaHCO₃ solution (25 ml) and [lacuna] stirred vigorously until a clear solution has formed. The aqueous phase is extracted with CH₂Cl₂ (2×25 ml). The combined organic phases are dried (MgSO₄) and concentrated under vacuum. After purification by flash chromatography (petroleum ether/EtOAc 5:1), the aldehyde 17 (1.41 g, 81%) is obtained as a colourless liquid. The aldehyde 17 decomposes very rapidly and is therefore used immediately in the following Still-Gennari olefination.

### Example 16

### Still-Gennari Olefination of the Aldehyde 17 to Give the Ester 18

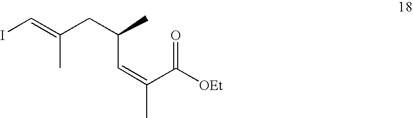

Still-Gennari reagent (2.90 g, 8.38 mmol) in 10 ml THF is added to a solution (−40° C.) of 18-crown-6 (3.51 g, 13.3 mmol) in 30 ml THF. The solution is cooled to −78° C. and a KHMDS solution (16.0 ml, 0.5 M in toluene, 8 mmol) is added slowly dropwise using a syringe. After 15 min the aldehyde 17 from the previous reaction (1.59 g, 6.68 mmol) dissolved in 10 ml THF is added dropwise. The reaction is quenched by adding saturated aqueous NaHCO₃ solution [lacuna] extracted with MTBE and dried with MgSO₄. The filtrate is concentrated under vacuum and purified by column chromatography (petroleum ether/EtOAc 3:1). The ester 18 (1.73 g, 85%) is obtained as a colourless oil. R_{f}=0.51 (petroleum ether/EtOAc 4:1); [α]²⁰_{D}−32.4 (c 1.0, CHCl₃); ¹ H-NMR (400 MHz, CDCl₃) δ 5.83 (d, J=0.8 Hz, 1H), 5.58 (dd, J=9.8, 1.2 Hz, 1H), 4.17 (q, J=7.1 Hz, 2H), 3.30-3.40 (m, 1H), 2.20 (dd, J=13.5, 6.9 Hz, 1H), 2.10 (dd, J=13.5, 7.5 Hz, 1H), 1.85 (d, J=1.2 Hz, 3H), 1.78 (d, J=0.7 Hz, 3H), 1.28 (t, J=7.1 Hz, 3H), 0.90 (d, J=6.7 Hz, 3H); ¹³C-NMR δ 167.9, 147.0, 146.3, 126.5, 75.8, 60.2, 47.0, 31.5, 23.8, 20.8, 19.9, 14.3; IR 2977, 1712, 1454, 1372 cm⁻¹; MS (EI) m/z (%)=322 (5) [M]⁺, 195 (25), 121 (48), 113 (100), 95 (22), 67 (15); HRMS calculated for C₁₂H₁₉10₂: 322.0430, found 322.0431.

### Example 17

### Reduction of the Ester 18 to Give the Alcohol 19

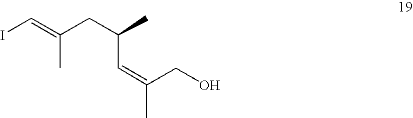

Diisobutylaluminium hydride (6.0 ml, 1.2 M in toluene) is added slowly dropwise to a solution (−78° C.) of the ester 18 (635 mg, 1.97 mmol) in 5 ml CH₂Cl₂. The solution is stirred for one hour at this temperature and diluted with 15 ml MTBE. After adding 1 ml H₂O the mixture is stirred vigorously until a white gel has formed. NaOH (2.5 ml, 4M) and H₂O (1 ml) are added to this gel and the suspension is stirred until a white solid has formed. The mixture is dried with MgSO₄ and the solids are separated off by filtration. The filtrate is concentrated under vacuum and purified by flash chromatography (petroleum ether/EtOAc 4:1). Alcohol 19 (425 mg, 77%) is obtained as a colourless oil: R_{f}=0.15 (petroleum ether/EtOAc 4:1); [α]²⁰_{D}−3.7 (c 1.0, CHCl₃); ¹ H-NMR (400 MHz, CD₃OD) 6), 5.80 (d, J=0.9 Hz, 1H), 4.99 (d, J=9.6 Hz, 1H), 4.00-4.10 (m, 2H), 2.55-2.70 (m, 1H), 2.05-2.15 (m, 2H), 1.78 (d, J=0.9 Hz, 3H), 1.75 (d, J=1.2 Hz, 3H), 0.89 (d, J=6.6 Hz, 3H); ¹³C-NMR (100 MHz, CD₃OD) δ 146.5, 133.8, 133.6, 75.9, 61.8, 47.6, 30.7, 24.1, 21.2, 21.1; IR 3316, 2956, 2921, 1867, 1452, 1376, 1273, 1000 cm⁻¹; MS (EI) m/z (%)=262 (20), 153 (15), 135 (14), 99 (100), 95 (49), 81 (16); HRMS calculated for C₁₀H₁₇IO: 280.0324, found 280.0324.

### Example 18

### Bromination of the Alcohol 19 to Give the Bromide 20

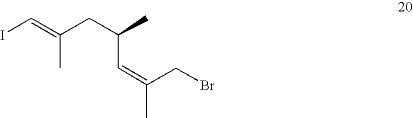

The alcohol 19 (140 mg, 0.5 mmol) is dissolved in 5 ml acetonitrile at room temperature. Triphenylphosphane (262 mg, 1 mmol) and CBr₄ (331 mg, 1 mmol) are added successively and the suspension thus obtained is stirred for 10 min at room temperature. The reaction is quenched with 2 ml H₂O and [lacuna] extracted with petroleum ether. The combined organic phases are dried with MgSO₄ and concentrated under vacuum. After filtering through a short silica gel column with petroleum ether the bromide 20 (120 mg, 70%) is obtained as a colourless liquid: ¹ H-NMR (400 MHz, CDCl₃) 65.85 (d, J=1.0 Hz, 1H), 5.10 (dq, J=9.8, 1.5 Hz, 1H), 3.92 (d, J=3.0 Hz, 2H), 2.61 (dq, J=9.8, 6.8 Hz, 1H), 2.16 (dt, J=7.2, 1.1 Hz, 2H), 1.81 (d, J=1.1 Hz, 3H), 1.80(d, J=1.5 Hz, 3H), 0.92 (d, J=6.6 Hz, 3H);); (sic) ¹³C-NMR (100 MHz, CDCl₃) δ 146.0, 136.5, 130.8, 76.2, 47.0, 32.1, 31.2, 24.1, 21.9, 20.2;

### Example 19

### Conversion of the Bromide 20 to the Phosphonium Salt B1

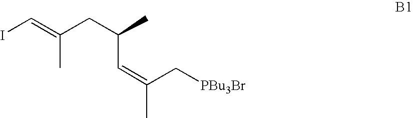

Tributylphosphane (106 mg, 0.13 ml, 0.52 mmol) is added slowly dropwise to a solution of the bromide 20 from the previous reaction (120 mg, 0.35 mmol) in 3 ml acetonitrile. The solution is stirred for 2 h at room temperature and then concentrated under vacuum. The phosphonium salt B1 is obtained as a brown oil that is used without further purification in the Wittig reaction with the C fragment.

### Example 20

### Hetero-Diels-Alder Reaction to Give the Ester 21

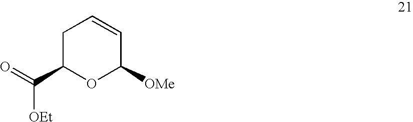

A solution of Ti(OiPr)₄ (0.223 ml, 0.75 mmol) in 1 ml CH₂Cl₂ is added dropwise to a solution of (R)-BINOL (429 mg, 1.5 mmol) in 2 ml CH₂Cl₂. The mixture is heated for 1 h under reflux and then cooled to −30° C. Freshly distilled ethyl glyoxylate (870 mg, 7.5 mmol) is added first of all, followed by a solution of 1-methoxy-1,3-butadiene (504 mg, 6.0 mmol) in 1 ml CH₂Cl₂. The solution is stirred for 2.5 h at

−30° C. and then heated to room temperature. The reaction is quenched with saturated aqueous NaHCO₃ solution and [lacuna] extracted with MTBE. The combined organic phases are dried with MgSO₄, filtered and concentrated under vacuum. After purification by flash chromatography (petroleum ether/ether, 8:1), the ester 21 is obtained as a colourless liquid (780 mg, 65%): R_{f}=0.34 (petroleum ether/EtOAc 4:1); [α]²⁰_{D}+45.6 (c 1.0, CHCl₃); ¹ H-NMR (400 MHz, CDCl₃) δ 6.01 (ddt, J=10.3, 4.0, 1.5 Hz, 1H), 5.67 (dq, J=10.3, 2.0 Hz, 1H) 5.15 (m, 1H), 4.36 (dd, J=6.5, 5.1 Hz, 1H), 4.12-4.27 (m, 2H), 3.48 (s, 3H), 2.42-2.52 (m, 1H), 2.27-2.36 (m, 1H), 1.28 (t, J=7.2 Hz, 3H); ¹³C-NMR δ 171.2, 127.6, 125.4, 95.9, 65.8, 61.1, 55.6, 27.5, 14.2; IR 2930, 1736, 1447, 1372 cm⁻¹; HRMS calculated for C₉H₁₄O₄: 186.0892, found 186.0830.

The corresponding enantiomer can be prepared by using (S)-BINOL.

### Example 21

### Reduction of the Ester 21 to Give the Alcohol 22

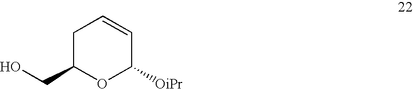

The ester 21 (100 mg, 0.54 mmol) is added using a syringe to a suspension of LiAlH₄ (20 mg, 0.54 mmol) in 10 ml Et₂O at 0° C. The suspension is stirred at this temperature for 45 min and then quenched by the successive addition of water (0.025 ml), 15% NaOH solution (0.025 ml) and water (0.050 ml) again. The aluminium salts are removed by filtration and the filtrate is concentrated under vacuum. After purification by flash chromatography (petroleum ether/Et₂O 1:1) an alcoholic intermediate is obtained as a colourless oil (80 mg, 100%). The intermediate (1.48 g, 10.27 mmol) is taken up in 20 ml iPrOH and 50 mg PPTS is added. The solution is stirred for 4 h at room temperature and quenched by adding saturated aqueous NaHCO₃ solution (100 ml) and EtOAc (100 ml). After separating the phases, the aqueous phase is extracted with EtOAc. The combined organic phases are dried with MgSO₄ and filtered and the filtrate is concentrated under vacuum. After purification by flash chromatography (petroleum ether/EtOAc 4:1) and recrystallisation from petroleum ether, the alcohol 22 is obtained as a colourless solid (1.37 g, 77%): R_{f}=0.10 (petroleum ether/EtOAc 6:1); [α]²⁰_{D}+33.2 (c 0.9, CHCl₃); ¹H-NMR δ 5.90-6.00 (m, 1H), 5.65-5.70 (m, 1H), 5.05-5.10 (m, IH), 4.00-4.10 (m, 1H), 3.97 (sep, J=6.3 Hz, 1H), 3.65-3.70 (m, 1H), 3.55-3.60 (m, 1H), 1.80-2.20 (m, 2H), 1.22 (d, J=6.3 Hz, 3H), 1.14 (d, J=6.1 Hz, 3H); 13C-NMR (100 MHz, CDCl₃) δ 128.1, 125.9, 92.7, 69.5, 66.6, 65.2, 25.9, 23.7, 21.9; IR (CHCl₃) 3296, 1657 cm⁻¹; HRMS calculated for C₉H₁₆O₃: 172.1099, found 172.1093.

### Example 22

### Oxidation of the Alcohol 22 to Give the Aldehyde C1

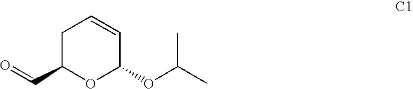

DMSO (0.25 ml) is added dropwise to a solution (−78° C.) of oxalyl chloride (0.18 ml, 2.06 mmol) in 2 ml CH₂Cl₂. After 5 min a solution of the alcohol 22 (300 mg, 1.76 mmol) in 4 ml CH₂Cl₂ and 0.13 ml DMSO is added dropwise. After the solution has been stirred for 40 min at −78° C., triethylamine (1.0 ml) is added slowly dropwise and the solution is heated to room temperature. The solvent is carefully removed in a rotary evaporator and the residue is chomatographed (CH₂Cl₂/MTBE 20:1). The aldehyde C1 (295 mg, 100%) is obtained as a colourless liquid: R_{f}=0.27 (CH₂Cl₂/MTBE 20:1); [α]²% D+90.0 (c 0.7, CDCl₃); ¹ H-NMR (400 MHz, CDCl₃) δ 9.70 (s, 1H), 5.95-6.00 (m, 1H), 5.70-5.75 (m, 1H), 5.20 (bs, 1H), 4.39 (dd, J=4.7, 11.3 Hz, 1H), 4.03 (sep, J=6.2 Hz, 1H), 2.10-2.30 (m, 2H), 1.22 (d, J=6.3 Hz, 3H), 1.17 (d, J=6.1 Hz, 3H); ¹³C-NMR (100 MHz, CDCl₃) δ 201.1, 126.8, 126.5, 93.0, 71.0, 53.4, 24.9, 23.7, 21.9; IR (CHCl₃) 2971, 2931, 1738, 1381, 1317, 1105, 1017 cm⁻¹; HRMS calculated for C₉H₁₄O₃: 171.1021, found 171.1015.

### Example 23

### Wittig Reaction of B1 and C1 to Give Compound 23

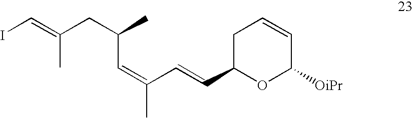

The aldehyde C1 (170 mg, 0.98 mmol) is added to a solution (0° C.) of the phosphonium salt B1 (370 mg, 0.81 mmol) in 8 ml toluene. KOtBu (1.10 ml, 1.10 mmol, 1.0 M in THF) is then added slowly dropwise using a syringe. After 15 min at this temperature the reaction is quenched with 2 ml water, [lacuna] extracted with ether and dried with MgSO₄. The solvent is removed under vacuum and the residue is purified by column chromatography (petroleum ether/EtOAc 8:1). Compound 23 (258 mg, 76%) is obtained as a colourless oil: R_{f}=0.20 (petroleum ether/EtOAc 1:1); [α]²⁰D+45.8 (c 1.0, CHCl₃); ¹ H-NMR (400 MHz, CD₃OD) δ 6.68 (d, J=15.7 Hz, 1H), 6.00-6.05 (m, 1H), 5.91 (d, J=1.0 Hz, 1H), 5.60-5.80 (m, 2H), 5.10-5.20 (m, 2H), 4.01 (sep, J=6.2 Hz, 1H), 2.80-2.95 (m, 1H), 2.20 (ddd, J=13.5, 6.5, 1.1 Hz, 2H), 2.05-2.10 (m, 2H), 1.80 (d, J=1.1 Hz, 3H), 1.79 (d, J=1.0 Hz, 3H), 1.17 (d, J=6.2 Hz, 3H), 1.23 (d, J=6.2 Hz, 3H), 0.94 (d, J=6.5 Hz, 3H); ¹³C-NMR (100 MHz, CD₃OD) δ 147.6, 137.5, 131.9, 130.8, 129.5, 129.0, 127.2, 94.8, 76.2, 71.2, 68.5, 48.2, 32.0, 31.4, 24.3, 24.2, 22.4, 21.1, 20.7; IR (CHCl₃): 1658, 1614, 1378, 1315, 1273, 1026, 998 cm⁻¹; HRMS (EI) calculated for C₁₉H₂₉10₂: 416.1212, found 416.1385.

### Example 24

### Heck Coupling of Compound 23 and Fragment A1 to Give the Acetal 24

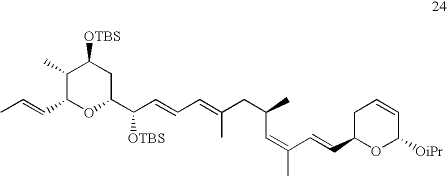

Bu₄NBr (70 mg, 0.22 mmol), Cs₂CO₃ (84 mg, 0.26 mmol), Pd(OAc)₂ (48 mg, 0.22 mmol) and Et₃N (0.026 ml, 0.19 mmol) are added successively to a solution of the A1 fragment (122 mg, 0.28 mmol) and compound 23 (73 mg, 0.17 mmol) in 0.3 ml DMF. The suspension is stirred for 36 h at room temperature and then chromatographed immediately on a silica gel column (petroleum ether/EtOAc 16:1). The Heck product (acetal) 24 (83 mg, 65%) is obtained as a colourless oil.

R_{f}=0.26 (petroleum ether/EtOAc 5:1); [α]²⁰D+49.5 (c 1.0, CHCl₃); ¹H-NMR (400 MHz, CD₃OD) δ 6.72 (d, J=15.8 Hz, 1H), 6.43 (ddd, J=15.2, 11.0, 1.6 Hz, 1H), 6.00-6.05 (m, 1H), 5.75 (d, J=11.0 Hz, 1H), 5.60-5.70 (m, 3H), 5.49 (dd, J=15.0, 5.0 Hz, 1H), 5.41 (ddd, J=15.5 Hz, 5.6 Hz, 1.6 Hz, 1H), 5.10-5.30 (m, 2H), 4.40-4.50 (m, 1H), 4.34-4.38 (m, 1H), 4.22-4.28 (m, 1H), 4.01 (sep, J=6.1 Hz, 1H), 3.90-3.95 (m, 1H), 3.66 (ddd, J=11.5, 4.0, 2.0 Hz, 1H), 2.80-2.90 (m, 1H), 2.00-2.10 (m, 4H), 1.78 (d, J=1.4 Hz, 3H), 1.72 (d, J=1.1 Hz, 3H), 1.69 (ddd, J=6.5, 1.5, 1.4 Hz, 3H), 1.35-1.60 (m, 3H), 1.23 (d, J=6.2 Hz, 3H), 1.17 (d, J=6.2 Hz, 3H), 0.93 (d, J=6.5 Hz, 3H), 0.91 (2 s, 18H), 0.86, (d, J=7.2 Hz, 3H), 0.09 (s, 3H), 0.06 (s, 3H), 0.05 (s, 3H), 0.04 (s, 3H); ¹³C-NMR (100 MHz, CD₃OD) δ 138.5, 137.6, 132.4, 132.3, 131.2, 130.4, 129.5, 129.3, 128.3, 127.2, 127.1, 126.3, 94.9, 77.5, 77.0, 76.1, 72.3, 71.2, 68.6, 41.7, 32.0, 31.3, 29.2, 28.0, 26.5, 26.4, 24.3, 22.5, 21.3, 20.8, 19.2, 19.0, 18.1, 16.9, 11.6, −4.1, −4.3, −4.6, −4.7; HRMS calculated for C₄₃H₇₆O₅Si₂: 729.5231, found 729.5236.

### Example 25

### Oxidation of the Acetal 24 to Give the Lactone 25

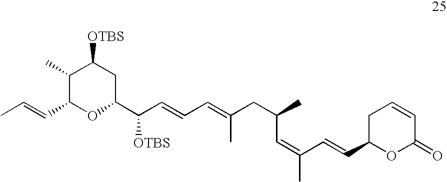

PPTS (6 mg) is added to a solution of the acetal 24 (20 mg, 0.03 mmol) in 3 ml acetone and 0.5 ml water. The solution is stirred for 12 h at room temperature and then quenched with saturated aqueous NaHCO₃ solution. The aqueous phase is extracted with EtOAc, the combined organic phases are dried with MgSO₄ and filtered and the filtrate is concentrated under vacuum. After purification by flash chromatography a lactol (16 mg, 83%) is obtained as a colourless oil. The lactol is taken up in 2 ml CH₂Cl₂ and MnO₂ (20 mg) is added. The suspension is stirred for 12 h at room temperature and then immediately introduced into a silica gel column. By means of flash chromatography (petroleum ether/EtOAc 5:1) the lactone 25 (12 mg, 79%) is obtained as a colourless oil: Rf=0.19 (petroleum ether/EtOAc 5:1); [α]²⁰_{D}+40.0 (c 0.2, CHCl₃); ¹ H-NMR (400 MHz, CD₃OD) δ 7.04 (ddd, J=9.8, 5.5, 2.9 Hz, 1H), 6.82 (d, J=15.7 Hz, 1H), 6.44 (ddd, J=15.2, 11.0, 1.5 Hz, 1H), 6.02 (ddd, J=9.8, 2.8, 1.3 Hz, 1H), 5.70-5.80 (m, 2H), 5.64 (ddd, J=15.5, 6.5, 1.5 Hz, 1H), 5.50 (dd, J=15.2, 5.8 Hz, 1H), 5.41 (ddd, J=15.5, 5.6, 1.6 Hz, 1H), 5.20-5.30 (m, 1H), 5.00-5.10 (m, 1H), 4.35-4.40 (m, 1H), 4.20-4.30 (m, 1H), 3.90-3.95 (m, 1H), 3.67 (ddd, J=11.6, 3.9, 2.2 Hz, 1H), 2.852.95 (m, 1H), 2.40-2.60 (m, 2H), 2.00-2.10 (m, 2H), 1.80 (d, J=1.3 Hz, 3H), 1.73 (d, J=1.1 Hz, 3H), 1.69 (ddd, J=6.5, 1.4, 1.3 Hz, 3H), 1.30-1.60 (m, 3H), 0.901.00 (m, 6H), 0.92 (s, 9H), 0.90 (s, 9H), 0.09 (s, 3H), 0.06 (s, 3H), 0.05 (s, 3H), 0.04 (s, 3H); ¹³C-NMR (100 MHz, CD₃OD) δ 166.6, 140.2, 147.9, 137.6, 132.5, 132.3, 131.2, 130.8, 128.4, 127.3, 127.2, 126.4, 121.6, 80.1, 77.5, 77.0, 76.1, 72.4, 41.8, 31.4, 31.0, 29.3, 26.5, 26.3, 21.2, 20.6, 19.2, 18.9, 18.0, 16.9, 11.6, 4.1, −4.3, −4.6, −4.7; HRMS calculated for C₄₀H₆₈O₅Si₂: 685.4605, found 685.4608.

### Example 26

### DeDrotection of Compound 25 to Give the Dreferred Ratiadon Derivative 1

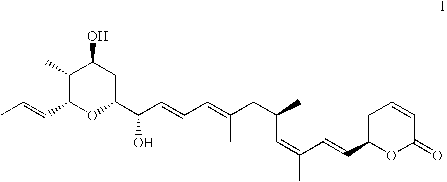

HF-pyridine (0.2 ml) is added dropwise at room temperature to a solution of the lactone 25 from the previous reaction (4 mg, 6 mmol) in 0.3 ml THF and 0.3 ml pyridine. The solution is stirred at room temperature for 24 h and quenched with saturated aqueous NaHCO₃ solution. This mixture is taken up in EtOAc and phosphate buffer (pH 7). The phases are separated and the organic phase extracted with EtOAc. The combined organic phases are dried with MgSO₄ and filtered and the filtrate is concentrated under vacuum. After purification by flash chromatography (CH₂Cl₂/CH₃OH, 16:1) ratjadon derivative 1 (2.0 mg, 76%) is obtained as a colourless solid: R_{f}=0.25 (CH₂Cl₂/methanol 19:1); [α]²⁰_{D}+48.0 (c 0.1, CHCl₃); ¹ H-NMR (400 MHz, CD₃OD) δ 7.05 (ddd, J=9.6, 5.5, 2.9 Hz, 1H), 6.46 (ddd, J=15.2, 10.9, 1.2 Hz, 1H), 6.01 (ddd, J=9.8, 2.4, 1.3 Hz, 1H), 5.805.85 (m, 1H), 5.68 (ddq, J=15.5, 6.5, 1.3 Hz, 1H), 5.62 (dd, J=15.2, 6.7 Hz, 1H), 5.46 (ddd, J=15.5, 6.0, 1.6 Hz, 1H), 5.20-5.30 (m, 1H), 5.05-5.15 (m, 1H), 4.354.40 (m, 1H), 4.06 (ddd, J=6.5, 2.5, 1.2 Hz, 1H), 3.85-3.90 (m, 1H), 3.76 (ddd, J=12.2, 4.2, 2.5 Hz, 1H), 2.40-2.60 (m, 2H), 2.85-2.95 (m, 1H), 1.79 (d, J=1.6 Hz, 3H), 1.95-2.10 (m, 2H), 1.74 (d, J=1.3 Hz, 3H), 1.69 (ddd, J=6.5, 1.5, 1.3 Hz, 3H), 1.80-1.85 (m, 1H), 1.60-1.65 (m, 1H), 1.45-1.50 (m, 1H), 0.94 (d, J=6.3 Hz, 3H), 0.88 (d, J=7.1 Hz, 3H); ¹³C-NMR (CD₃OD) δ 166.7, 148.0, 140.1, 138.1, 132.0, 131.3, 130.9, 129.3, 127.5, 127.1, 126.9, 121.5, 80.1, 76.8, 76.1, 76.0, 71.0, 40.7, 31.7, 31.0, 29.2, 21.3, 20.6, 18.0, 17.0, 11.6; HRMS calculated for C₂₈H₄₀O₅: 456.2876, found 456.2880.

### Example 27

### Synthesis of the Aldehyde 27 from the Diol 26

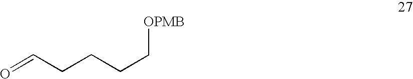

The diol 26 (8.17 g, 78.44 mmol) in 12 ml dry toluene is initially introduced into a 50 ml round-bottomed flask equipped with an argon balloon flask and reflux condenser. The solution is stirred and sodium hydride (60% suspension in mineral oil) (1.53 g, 38.25 mmol) is added thereto in portions over a period of 45 min. The suspension is heated under reflux for 3 h under an argon blanketing gas atmosphere. 4-Methoxy-benzyl chloride (5.2 ml, 38.31 mmol) is then added over a period of 30 min and the mixture is boiled under reflux for 18 h. At the end of this period the reaction mixture is cooled to room temperature and poured into 50 ml water. The phases are separated and the aqueous phase is extracted with dichloromethane (3×50 ml). The combined organic phases are dried over Na₂SO₄ and concentrated under vacuum. After purification on silica gel (EtOAc:petroleum ether=1:1), 5-(4-methoxy-benzyloxy)-pentan-1-ol (3.469 g) is obtained as a colourless oil (40% yield): R_{f}=0.32 in hexane:EtOAc (2:3); ¹H NMR (200 MHz, CDCl₃)(δ ppm): 1.46-1.60 (m, 6H), 3.45 (t, 2H), 3.60 (t, 2H), 3.80 (s, 3H), 4.43 (s, 2H), 6.89 (dd, 2H), 7.26 (dd, 2H).

The compound thus obtained is then used in the next reaction.

Oxalyl chloride (1.45 g, 16.62 mmol) and 75 ml dry dichloromethane are filled into a 50 ml round-bottomed flask equipped with an argon balloon flask and reflux condenser. The solution is cooled to −78° C. and a solution of DMSO (2.36 ml, 33.25 mmol) in 5 ml dry dichloromethane is added thereto over a period of 5 min. The reaction [lacuna] is stirred for a further 15 min. A solution of 5-(4methoxy-benzyloxy)-pentan-1-ol (3.4 g, 15.16 mmol) from the synthesis reaction described above in 5 ml dry dichloromethane is then added and the reaction mixture is stirred at −78° C. for 2 h. Triethylamine (10.56 ml, 75.76 mmol) is added to the solution and the reaction mixture is stirred at room temperature (RT, approx. 20° C.) for 1 h. The reaction is discontinued by adding 150 ml water and the aqueous phase is extracted with dichloromethane (3×50 ml). The combined organic phases are washed with saturated NaCl solution (2×100 ml) and dried over Na₂SO₄. After filtering off the desiccant, the solution is concentrated under vacuum and purified with silica gel (hexane:EtOAc=8:2). 3.36 g of the aldehyde 27 are thus obtained as a colourless oil (98% yield): ¹H NMR (200 MHz, CDCl₃) (6 ppm): 1.66-1.70 (m, 6H), 2.45 (m, 2H), 3.46 (m, 2H), 3.81 (m, 2H), 4.42 (s, 2H), 6.90 (d, 2H), 7.27 (m, 2H), 9.76 (s, 1H).

### Example 28

### Wittig-Horner Reaction of the Aldehyde 27 to Give the Ester 28

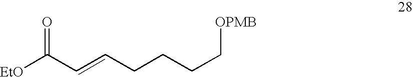

Sodium hydride (60% suspension in mineral oil) (0.60 g, 24.70 mmol) and 49 ml dry THF are introduced into a 100 ml two-necked round-bottomed flask equipped with an argon blanketing gas balloon flask. The contents are stirred and cooled to 0° C. Triethyl phosphonoacetate (3 ml, 14.84 mmol) is then added slowly dropwise and the mixture is stirred for 90 min at 0° C. A solution of aldehyde 27 (3 g, 13.49 mmol) in THF (14 ml) is added slowly dropwise to this solution at 0° C. When the addition is complete, the ice bath is removed and the reaction mixture is stirred at RT. After approx. 2 h the reaction is discontinued by adding saturated NH₄Cl solution. The phases are separated and the aqueous phase is extracted with ethyl acetate (3×50 ml). After purifying by silica gel flash chromatography, ester 28 (1.909) is obtained as a colourless oil (60% yield): R_{f}=0.46 in hexane:EtOAc (2:3); ¹H NMR (200 MHz, CDCl₃) (δ ppm): 1.25-1.32 (m, 6H), 2.212.23 (m, 2H), 3.41 (m, 3H), 3.81 (m, 4H), 4.16 (m, 4H), 4.43 (s, 2H), 5.77-5.85 (d, 1H, Ha, J=15.6 Hz), 6.90 (m, 3H, Hb, J=8.66 Hz), and 7.26 (dd, 2H).

### Example 29

### Reduction of the Ester 28 to Give the Allyl Alcohol 29

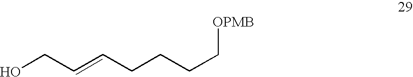

Ester 28 (10 g, 34.24 mmol) and 170 ml dry dichloromethane are introduced into a 250 ml two-necked round-bottomed flask equipped with an argon blanketing gas balloon flask. The solution is cooled to −78° C. Dibutylaluminium hydride (1 M in hexane) (97.6 ml, 97.58 mmol) is then added dropwise and the reaction mixture is stirred for 1 h. The reaction solution is diluted with 70 ml MTB ether and the reaction is discontinued by adding 12 ml water. The resulting solution is stirred vigorously at RT until a white precipitate forms. 4N NaOH (12 ml) and water (24 ml) are added to this mixture. The suspension is stirred. The organic phase is separated off, dried over MgSO₄ and concentrated under vacuum. After purifying by silica gel flash chromatography (hexane:ethyl acetate=7:3), allyl alcohol 29 (6.3 g) is obtained as a colourless oil (74% yield): R_{f}=0.46 in hexane:EtOAc (1:1); ¹H NMR (200 MHz, CDCl₃) (5 ppm): 1.53 (m, 6H), 2.04 (m, 2H), 3.44 (t, 2H), 3.80 (s, 3H), 4.07 (d, 2H), 4.42 (s, 2H), 5.65 (m, 2H), 6.85-6.90 (d, 2H), and 7.24-7.28 (d, 2H).

### Example 30

### Asymmetric Sharpless Epoxidation of the Allyl Alcohol 29 to Give the Epoxide 30

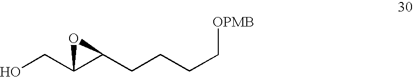

Activated molecular sieve (4 Å) (3.6 g) and 42 ml dry dichloromethane are introduced into a 100 ml two-necked, round-bottomed flask equipped with an argon blanketing gas balloon flask. The reaction solution is cooled to −25° C. L (+) DET (0.350 ml, 2.03 mmol) and Ti(iOPr)₄ (0.416 ml, 1.317 mmol) are added successively to the stirred solution. TBHP (5.5 M in decane) (5.01 ml, 27.52 mmol) is then added dropwise over a period of 10 min and the reaction mixture is stirred for 30 min. A solution of the allyl alcohol 29 (3 g, 12 mmol) in 10 ml dry dichloromethane is then added dropwise over a period of 30 min and the reaction mixture is stirred for 3 h at a temperature of −20° C. The reaction is discontinued by adding water (8.4 ml) and [lacuna] is then stirred for 30 min at RT. 30% sodium hydroxide solution (0.8 ml) is then added and the reaction mixture is stirred for a further 30 min. The crude product is extracted with dichloromethane (4×150 ml). The organic phases are combined and dried over Na₂SO₄. After concentrating under vacuum, purification is carried out by silica gel flash chromatography (hexane:EtOAc=8:2). Epoxide 30 (2.186 g) is thus obtained as a colourless oil (68.4% yield): R_{f}=0.20 in hexane:EtOAc (1:1); [α]_{D}²⁰=−15.8 (c=1, chloroform); ¹H NMR (400 MHz, CDCl₃) (δ ppm): 1.57 (m, 6H), 2.89-2.90 (m, 2H), 3.44 (t, 2H), 3.61 (dd, 1H), 3.79 (s, 3H), 3.68 (dd, 1H), 4.42 (s, 2H), 6.88 (d, 2H), 7.24-7.25 (m, 2H); ¹³C NMR (100 MHz, CDCl₃) (δ ppm): 22.68, 29.43, 31.32, 55.24, 55.79, 58.33, 61.64, 69.71, 72.56, 113.74, 129.22, 159.11.

### Example 31

### Cyclisation of the Epoxide 30 to Give the Diol 31

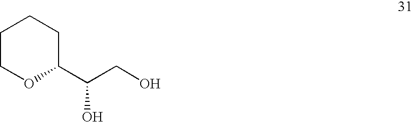

A solution of epoxide 30 (2.03 g, 7.63 mmol) in 48 ml dichloromethane/water (10:1) is introduced into a 50 ml two-necked, round-bottomed flask equipped with an argon blanketing gas balloon flask and the solution is cooled to 0° C. DDQ (2.87 g, 13.10 mmol) is added in portions to this solution. When the addition is complete the cooling bath is removed and the reaction [lacuna] is stirred for a further 3 h at RT. The reaction [lacuna] is then diluted with 100 ml dichloromethane. The organic phase is separated off and washed successively with 50 ml saturated NaCl solution, 50 ml saturated NaHCO₃ solution and again with 50 ml saturated NaCl solution. The organic phase is dried over Na₂SO₄ and filtered off from the desiccant. After concentration under vacuum, purification is carried out by silica gel flash chromatography (hexane:EtOAc=3:1). Diol 31 (0.180 g) is obtained as a colourless oil (14% yield): R_{f}=0.13 in hexane:EtOAc (2:3); [α]_{D}²⁰=−2.4 (c=1, chloroform); ¹H NMR (400 MHz, CDCl₃) (δ ppm): 1.35-1.66 (m, 6H), 1.83-1.86 (d, 1H), 2.73 (s, 2H), 3.38 (m, 2H), 3.58 (m, 1H), 3.66-3.70 (m, 2H), 3.98 (dd, 1H); ¹³C NMR (100 MHz, CDCl₃) (δ ppm): 23.01, 26.04, 27.47, 63.55, 68.77, 73.65, 79.98.

### Example 32

### Double TBS protection of the diol 31 to give the TBS ether 32

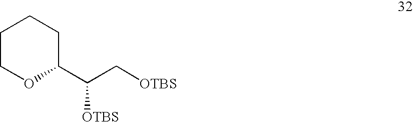

A solution of diol 31 (0.170 g, 1.16 mmol) in 8 ml dry dichloromethane is introduced into a 25 ml two-necked, round-bottomed flask equipped with an argon blanketing gas balloon flask and cooled to 0° C. 2,6-Lutidine (0.693 ml, 5.822 mmol) is then added slowly. TBMSOTf (0.802 ml, 3.493 mmol) is then added dropwise to this solution over a period of 30 min. When the addition is complete the cooling is removed and the reaction mixture is stirred for 12 h at RT. The reaction is then discontinued by adding saturated NaHCO₃ solution (5 ml) and water (9 ml). The aqueous phase is extracted with dichloromethane (4×5 ml). The combined organic phases are dried over Na₂SO₄. After concentration under vacuum, and purification by silica gel flash chromatography (hexane:EtOAc=3:1). 0.400 g of the TBF ether 32 is obtained as a colourless oil (92% yield): R_{f}=0.79 in hexane:EtOAc (3:2); [α]D 20=5.2 (c=1, chloroform); ¹H NMR (200 MHz, CDCl₃) (δ ppm): −0.082 (s, 6H), 0.84 (m, 18H), 1.23 (m, 5H), 2.01 (s, 1H), 3.56 (m, 1H), 4.09 (m, 1H).

### Example 33

### Deprotection of the Primary OH Group in the TBS Ether 32 to Give the Mono-Protected Alcohol 33

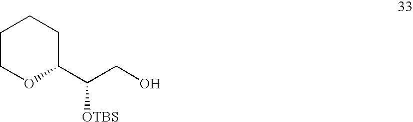

TBS ether 32 (0.144 g, 0.385 mmol) is dissolved in 2 ml ethanol and the solution is stirred at RT. PPTS (0.029 g, 0.115 mmol) is added to this solution and the mixture is heated to 65° C. After 2 h the reaction is complete and the solvent is removed under vacuum. The crude product is then purified by silica gel flash chromatography (hexane:EtOAc=4:1). 0.095 g of the alcohol 33 (95% yield) is obtained: R_{f}=0.70 in hexane:EtOAc (2:3); [α]_{D}²⁰=1.2 (c=1, chloroform); 1H NMR (400 MHz, CDCl₃) (δ ppm): 0.06 (s, 6H), 0.86 (s, 9H), 1.23 (ddd, 1H), 1.48 (m, 3H), 1.78 (m, 2H9, 2.23 (br, OH), 3.31-3.40 (m, 2H), 3.59-3.64 (m, 2H), 3.93 (dd, 1H); ¹³C NMR (100 MHz, CDCl₃) (δ ppm): −5.59, 17.03, 22.05, 24.81, 25.07, 27.68, 64.21, 67.53, 73.58, 78.97.

### Example 34

### Dess-Martin Oxidation of the Alcohol 33 to Give the Aldehyde 34

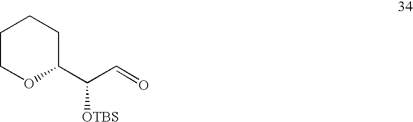

Dess-Martin reagent (0.218 g, 0.516 mmol) is added to a solution of the alcohol 33 (0.112 g, 0.430 mmol) in dichloromethane (13 ml) at 0° C. When the addition is complete the cooling is removed and the reaction mixture is stirred for 2 h at RT. The reaction is then discontinued by adding saturated NaHCO₃ solution (10 ml). The phases are separated and the organic phase is extracted with dichloromethane (3×10 ml). The combined organic phases are dried over Na₂SO₄ and concentrated under vacuum. After purification by silica gel chromatography (hexane:EtOAc=95:5), the aldehyde 34 (0.105 g) (94.5% yield) is obtained: R_{f}=0.6 in hexane:EtOAc (8:2).

This aldehyde is used immediately in the next reaction.

### Example 35

### Tebbe Olefination of the Aldehyde 34 to Give the Olefin 35

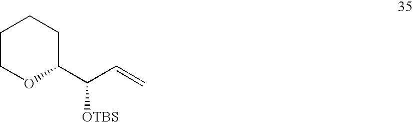

Tebbe reagent (0.830 ml, 0.407 mmol) is added to a solution of aldehyde 34 (0.105 g, 0.407 mmol) in THF (13 ml) at 0° C. The reaction solution is stirred under an Ar atmosphere for 30 min. At the end of this time the reaction is discontinued by adding MTB ether (20 ml) and 1M NaOH solution (0.2 ml). The organic phase is dried over Na₂SO₄, filtered off from the solvent and concentrated under vacuum. After purification by silica gel flash chromatography (hexane:EtOAc=9:1), the olefin 35 (0.093 g) (89.42% yield) is obtained: R_{f}=0.61 in hexane:EtOAc (9:1); [α]_{D}²⁰=−1.0 (c=1, chloroform); ¹H NMR (200 MHz, CDCl₃): 0.04 (d, 6H), 0.85 (s, 9H), 1.21-1.40 (m, 6H), 3.14 (m, 1H), 3.36 (m, 1H), 3.94 (m, 2H), 5.14-5.26 (dd, 2H), 5.70-5.81 (m, 1H).

### Example 36

### Influence of the Substance 1 According to the Invention on the Cell Cycle of Glioblastoma and HepG2 Cells

Glioblastoma cells and HepG2 cells (liver carcinoma cells) were in each case incubated for 48 h without the substance 1 according to the invention and in nutrient medium containing various concentrations of the substance 1 according to the invention. The cells were harvested and measured by continuous flow cytometry.

The results of the continuous flow cytometric measurements are shown in Figures Error! Bookmark not defined. to Error! Bookmark not defined. for glioblastoma cells and Error! Bookmark not defined. to Error! Bookmark not defined. for HepG2 cells, in each case in plot form. In each case the DNA content is indicated on the X axis and the cell count is indicated on the Y axis. The results shown in Figures Error! Bookmark not defined. to Error! Bookmark not defined. were obtained in each case with 0 nM, 10 nM and 20 nM, respectively, of the preferred substance 1 according to the invention in the culture medium; the results shown in Figures Error! Bookmark not defined. to Error! Bookmark not defined. were obtained with 0 nM, 2 nM and 5 nM, respectively, of the preferred substance 1 according to the invention in the culture medium.

It can be seen that for both cell lines tested the absolute number and the number of cells in the S and in the G₂ phase decreases with increasing concentration of substance 1. At a concentration of 20 nM (glioblastoma cells) and, respectively, 5 nM (HepG2), virtually all cells tested are in the G₁ phase. The preferred substance 1 according to the invention thus arrests the cell cycle in the G₁ phase at the dosage suitable for the particular cell type.

In addition, the shape of glioblastoma cells growing on a conventional carrier as a function of the concentration of the substance 1 according to the invention was investigated (no figure). In the absence of substance 1 the cells have their typical dendritic shape and adhere to to (sic) the carrier surface. In the presence of 50 nM of the preferred substance 1 the cells have largely lost their dendritic shape and detach easily from the carrier surface.

### Example 37

### Influence of the Substances 1.36 and 37 According to the Invention on the Growth of Tumor Cells

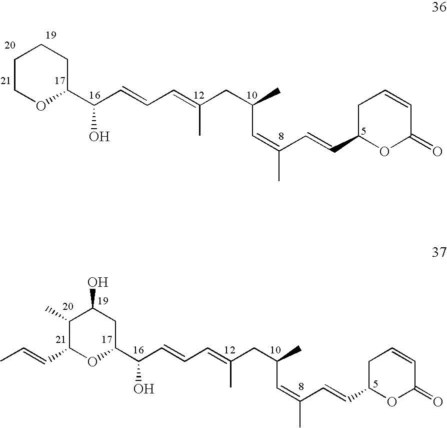

The tables below show the influence of the substances 1, 36 and 37 according to the invention on the growth of tumor cells, specifically the growth of the cell lines HM02 (gastroadenocarcinoma), HepG2 (liver carcinoma) and MCF 7 (mammary carcinoma).

The studies were carried out in accordance with the NCI guidelines (Grever et al., Seminars in Oncology 19 (1992), 622-638). The cells were cultured in 96-well microtitre plates in RPMI 1640 medium with 10% FCS (foetal calf serum). 24 h after dissemination of the cells, the substances 1 and 36 according to the invention dissolved in methanol were added and the cells were incubated for a further 48 h. The cell count was then determined.

In the tables, in each case:

GI₅₀: denotes the concentration that effects a semi-maximum inhibition of the cell growth

TGI: denotes the concentration that effects complete inhibition of the cell growth

LC₅₀: denotes the concentration that effects a semi-maximum cytotoxic effect, i.e. at which the cell count present 24 h after dissemination is reduced by half.

Cell line HMO2:

| Substance | GI₅₀ [ng/ml] | TGI [ng/ml] | LC₅₀ [ng/ml] |
|---|---|---|---|
| 1 | <5 | 24 | 250 |
| 36 | 5 | 140 | >500 |
| 37 | 17 | >100^{a} | >100 |

^{a}90% growth inhibition at 100 ng/ml

Cell line: HEP-G2

| Substance | GI₅₀ [ng/ml] | TGI [ng/ml] | LC₅₀ [ng/ml] |
|---|---|---|---|
| 1 | 13 | 500^{b} | >500 |
| 36 | 58 | >500^{c} | >500 |
| 37 | 85 | >100 | >100 |

^{b}80% growth inhibition at 500 ng/ml;
^{c}60% growth inhibition at 500 ng/ml

MCF 7

| Substance | GI₅₀ [ng/ml] | TGI [ng/ml] | LC₅₀ [ng/ml] |
|---|---|---|---|
| 1 | 16 | >500^{b} | <500 |
| 36 | 100 | >500^{c} | >500 |
| 37 | 25 | >100 | >100 |

^{b}80% growth inhibition at 500 ng/ml;
^{c}60% growth inhibition at 500 ng/ml

1. Substance of the formula 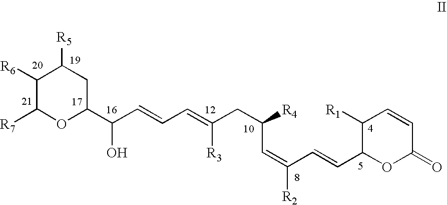
where
R₁, R₂ and R₃ independently of one another are selected from the group that consists of H, CH₃ and C₂H₅,
R₄ is CH₃ or C₂H₅,
R₅ is H or OH and
R₆ and R₇ independently of one another are selected from the group that consists of H, CH₃, C₂H₅, n-C₃H₇, 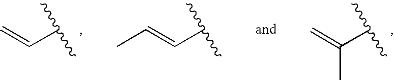
and where
C10 is R-configured and
C17 is R-configured if
(a) C16 is R-configured and at the same time
(b) neither R₅ nor R₆ nor R₇ is H.

2. Substance according to claim 1, where R₅, R₆ and R₇ are each H.

3. Formulation for inhibition of the reproduction of tumor cells, comprising
(a) a substance according to claim 1 or a mixture of two or more different substances according to claim 1 in a sufficient concentration to inhibit the reproduction of tumor cells, and
(b) a pharmaceutically acceptable excipient.

4. Use of a substance according to claim 1 or of a formulation according to claim 3 for the preparation of a medicament for inhibition of the reproduction of tumor cells.

5. Use of a substance according to claim 1 or of a formulation according to claim 3 for non-therapeutic inhibition of the reproduction of cells, in particular for non-therapeutic inhibition of the reproduction of tumor cells.

6. Method for non-therapeutic inhibition of the reproduction of tumor cells, characterised in that the tumor cells are exposed to a reproductioninhibiting dose of a substance according to claim 1 or to a reproductioninhibiting dose of a mixture of two or more different substances according to claim 1.

7. Substance of the formula 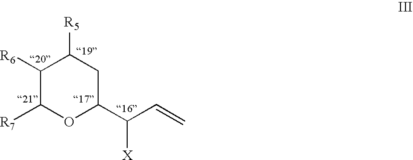
where
X is an unprotected or protected hydroxyl group,
R₅ is H, an unprotected or a protected hydroxyl group and
R₆ and R₇ independently of one another are selected from the group that consists of H, CH₃, C₂H₅, n-C₃H₇, iso-C₃H₇, 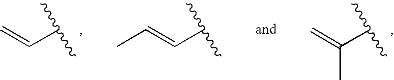
and where
C“17” is R-configured if
(a) C“16” is R-configured and at the same time
(b) neither R₅ nor R₅ nor R₇ is H.

8. Substance according to claim 7, where R₅, R₆ and R₇ are each H.

9. Method for the preparation of a substance according to claim 1, where a substance of the formula according to claim 7, in which, in each case, X is a protected hydroxyl group and R₅ is H or a protected hydroxyl group, is linked to an iodide of the formula 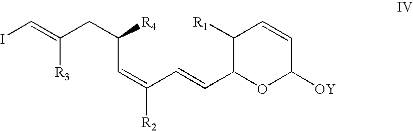
in which
R₁, R₂ and R₃ independently of one another are selected from the group that consists of H, CH₃ and C₂H₅
R₄ is CH₃ or C₂H₅ and
Y is methyl, ethyl or isopropyl.
